(19) United States
(12) Patent Application Publication
(10) Pub. No.: US 2019/0257832 A1
(43) Pub. Date: Aug. 22, 2019

(54) NATURAL MICROORGANISMS WHICH ARE NATURALLY CAPABLE OF BINDING TOXINS AND/OR TOXIN RECEPTORS

(71) Applicants: Philippe ULSEMER, Schildow (DE); Steffen GOLETZ, Glienicke-Nordbahn (DE); Peter GOETZ, Bubenreuth (DE)

(72) Inventors: Philippe ULSEMER, Schildow (DE); Steffen GOLETZ, Glienicke-Nordbahn (DE); Peter GOETZ, Bubenreuth (DE)

(21) Appl. No.: 16/318,532

(22) PCT Filed: Jul. 18, 2017
(86) PCT No.: PCT/EP2017/068115

§ 371 Date: Jan. 17, 2019

(30) Foreign Application Priority Data
Jul. 18, 2016 (EP) 16179883.0

(51) Int. Cl. G01N 33/569; C12N 1/16; C12N 1/20; A61P 1/00; A61K 35/74

(52) U.S. Cl. CPC G01N 33/56911; C12N 1/16; A61K 35/74; A61P 1/00; G01N 33/56961; C12N 1/20

(57) ABSTRACT

The present invention relates to means and method for isolating naturally-occurring microorganisms (non-pathogenic bacteria, yeasts or fungi) capable of binding toxins from microorganisms such as bacteria, viruses, fungi, yeasts, or protozoans and/or receptors for these toxins on the surface of mammalian cells, thereby making these receptors inaccessible for said toxins. The naturally-occurring microorganisms that are obtainable by the means and methods of the present invention can be used for adsorbing toxins from pathogenic microorganisms and/or blocking receptors for such toxins on the surface of mammalian cells. These toxin-receptor interactions are known to be critical for disease pathogenesis, making both the toxins and receptors a target for the naturally-occurring microorganisms of the present invention.

### CROSS-REFERENCE

This application is a national phase of International Application No. PCT/EP2017/068115, filed on Jul. 18, 2017, which claims priority to European Application No. 16179883.0, filed on Jul. 18, 2016, the entirety of each of which is herein incorporated by reference.

### FIELD OF THE INVENTION

The present invention relates to means and method for isolating naturally-occurring microorganisms (non-pathogenic bacteria, yeasts or fungi) capable of binding toxins from microorganisms such as bacteria, viruses, fungi, yeasts, or protozoans and/or receptors for these toxins on the surface of mammalian cells, thereby making these receptors inaccessible for said toxins. The naturally-occurring microorganisms that are obtainable by the means and methods of the present invention can be used for adsorbing toxins from pathogenic microorganisms and/or blocking receptors for such toxins on the surface of mammalian cells. These toxin-receptor interactions are known to be critical for disease pathogenesis, making both the toxins and receptors a target for the naturally-occurring microorganisms of the present invention. The present invention is not for use in the preparation of aliments, food supplements or products in accordance with the definition of the novel food regulation of the European Union.

### BACKGROUND OF THE INVENTION

For a number of infectious diseases, effective vaccines are missing and the increasing rate of drug resistances is complicating the use of conventional antimicrobial therapy. Due to this there is a need for novel therapeutic and prophylactic approaches against infectious diseases, in particular enteric infectious diseases which continue to cause massive morbidity and mortality in humans. Effective vaccines are still not available for a number of important diarrheal diseases, and, as mentioned, controlling these with conventional antimicrobial therapy is being complicated by increasing rates of drug resistance.

The initial and critical step that leads to an infection is the binding of a pathogen or its toxin(s) to a host cells (1). Anti-adhesion strategies aim to prevent and/or displace said binding and to prevent or treat the subsequent infection and/or its symptoms. Anti-adhesion strategies have attracted increasing interest as a source of novel therapeutics to prevent and treat infectious diseases. An advantage of such approaches is that the pathogen is not killed. As a consequence, anti-adhesion strategies may avoid problems associated with release of toxic products from dead bacteria and they may put much less selection pressure on pathogens, reducing the risk of resistance development.

Several anti-adhesion approaches may be envisaged, including providing receptor analogs or adhesin analogs, inhibition of adhesins and their host receptors, vaccination with adhesins or analogs, or inhibiting the synthesis of adhesins or their host receptor (2).

Although the initial adhesion of pathogens or their toxins to host cells may happen through protein-protein interactions (3, 4) or phospholipid-protein interactions (5) it is often mediated by protein-carbohydrate interactions (6, 7).

As a consequence, many carbohydrates or carbohydrate mimicking substances have been developed based either on proteins, polymers, calixarenes, dendrimers, cyclodextrins, cyclopeptides, fullerenes, gold nanoparticles and quantum dots (8). In order to efficiently block protein-carbohydrate interactions synthetic neutralization agents need to comprise multiple oligosaccharide epitopes displayed on complex three dimensional scaffolds, conditions that may be difficult to reproduce synthetically. However, to date, clinical results in human were disappointing, mostly because of toxicity or lack of efficacy [e.g. Synsorb PK (Synsorb Biotech); Tolevamer (Genzyme)].

Accordingly, U.S. Pat. No. 6,833,130 discloses recombinant microorganisms, genetically modified to express carbohydrate structures that mimic the natural binding moieties of bacterial toxins. Such microorganisms are able to present the binding moiety at high density. The efficacy of these microorganisms in binding toxins and protecting animals in lethal challenge models has been demonstrated (9, 10, 11, 12). However, in order to use microorganisms in humans and animals, it is not only necessary for these microorganisms to express a binding moiety for a pathogenic ligand at sufficient density, but it should preferably be harmless, ideally it should be non-pathogenic and not genetically modified or recombinant.

A limited amount of publications reports the ability of non-pathogenic microorganisms to specifically co-aggregate other microorganisms. However, the detailed mechanism of interaction is unknown (13, 14, 15). A few pathogenic microorganisms were described to naturally express structures that may mimic natural binding moiety for pathogenic ligands (16, 17, 18, 19).

Thus, as is evident from the above, the prior art provides recombinant microorganisms for use in treating infectious disease, particularly enteric infectious disease which may be harmful and which are not be food grade organisms. Alternatively, the prior art provides agents which have shown to be toxic when administered to mammals.

To summarize, up to now, to the best of the inventors' knowledge no non-pathogenic microorganisms naturally expressing binding structures that may, for example, be an analog to eukaryotic receptors for toxins have been described. However, it is highly desirable to block the interaction between pathogenic ligands such as toxins from pathogenic microorganisms and their cognate receptors on the surface of mammalian cells, since this interaction is crucial for disease pathogenesis. Such a strategy is particularly promising for fighting against enteric infectious diseases which cause a high morbidity and mortality in humans, since no effective vaccines are available and rates of drug resistance against conventional antibiotic therapies increase. Due to this there is a need for novel therapeutic and prophylactic approaches against infectious diseases, in particular enteric infectious diseases. It, thus, follows that the technical problem underlying the present invention is to comply with the needs described above. The solution to this technical problem is achieved by providing the embodiments characterized herein, exemplified in the appended examples and set out in the claims.

### SUMMARY OF THE INVENTION

Specifically, the present invention provides a solution for the need set out above and, thus, the object of the present invention is to provide novel strategies for the therapy and/or prevention of toxin-mediated infectious diseases, particularly enteric infectious diseases, which are suitable for human application. The present invention is not for use in the preparation of aliments, food supplements or products in accordance with the definition of the novel food regulation of the European Union.

More specifically, the present inventors developed a method that allows direct and selective isolation of naturally-occurring microorganisms (that are preferably non-pathogenic) that bind toxins from microorganisms such as pathogenic microorganisms and/or the corresponding toxin receptors present on mammalian cells, thereby competing with or blocking toxin and toxin receptor interaction that is known to be crucial for disease pathogenesis. Toxins from pathogenic microorganisms are known to recognize particularly oligosaccharides displayed on the surface of mammalian cells as receptors for toxins, while other toxins bind to as yet unknown receptors which can be identified by the means and methods of the present invention. In order to avoid genetic engineering of microorganisms, the methods of the present invention aim at the isolation of naturally-occurring microorganisms out of a mixture of naturally-occurring microorganisms that are present in, e.g. human samples, animal samples, soil, water, food or cultures of microorganisms.

Hence, the present invention preferably excludes the isolation and/or production of recombinant or genetically engineered microorganisms for the purpose of treating, alleviating or preventing an infectious disease, particularly an enteric infectious disease including a gastrointestinal disease as described herein.

The naturally-occurring microorganisms (obtained or obtainable by the methods of the present invention) or compositions comprising said microorganisms can be used to compete with or block the interaction between a toxin and its receptor, in order to treat, cure, abate or prevent infectious diseases of humans and/or animals. Furthermore the invention provides methods of reducing the amount of a toxin from an environment. By way of example, the present invention may be used for the detoxification of water, e.g. for removing bacterial toxins, such as cholera toxin, from water.

The present invention also provides naturally-occurring microorganisms or an analog, variant or fragment thereof all capable of binding a toxin from a microorganism such as a pathogenic microorganism and/or a surface receptor of a mammalian cell for a toxin from such a microorganism. Such a naturally-occurring microorganism, analog, variant or fragment, or lysates or fractions thereof, can be used for neutralizing a toxin and/or reducing the pathogenic effect of a pathogenic microorganism.

In addition, the present invention provides compositions comprising said naturally-occurring microorganism, analog, variant, fragment, lysates or fractions. Said analog, variant, fragment, lysates or fractions can bind a toxin from a microorganism such as a pathogenic microorganism and/or can bind a surface receptor of a mammalian cell for a toxin from a microorganism such as a pathogenic microorganism.

Furthermore, the present invention provides a method for treating, alleviating or preventing a gastrointestinal disease, comprising administering a therapeutically effective amount said naturally-occurring microorganism, analog, variant, fragment, lysates or fractions or said compositions.

Moreover, the present invention provides the use of said compositions for (use in a method of) treating, alleviating, or preventing a gastrointestinal disease. Finally the present invention provides a kit for performing a method for isolating a naturally-occurring microorganism that is capable of binding a toxin from a pathogenic microorganism and/or a surface receptor of a mammalian cell for a toxin from a pathogenic microorganism.

### BRIEF DESCRIPTION OF THE DRAWINGS

FIG. 1: Possible strategy for linking a carrier (magnet beads) and a binding molecule.

To isolate a microorganism naturally expressing a structure (A) that recognize a toxin, a binding molecule (defined as the toxin itself or any binding moiety (antibody, lectins etc. . . . ) that binds the structure (A)) may be linked to any carrier (e.g. magnet beads, . . . ) that can be washed out and isolated from a mixture of microorganisms.

[symbol] Biotin, [symbol] Tag (i.e. His Tag), [symbol] Ab @ binding molecule, [symbol] Toxin or Binding moiety.

FIG. 2: Binding of HRP-labeled heat labile toxin to GM1.

The binding of HRP-labeled heat labile toxin to wells previously coated with GM1 and pre-treated (+PJ) or not (−PJ) with periodate was analyzed by ELISA. Unspecific binding of HRP-LT was revealed on wells coated with PBS.

FIG. 3: Cytotoxic effect of LT and biotinylated LT on differentiated HT-29 cells.

HT-29 cells seeded at 2×10⁴ cells/well were cultured for 4 days in medium containing 2 mM butyrate. HT-29 cells were then incubated with PBS (A), 0.5 ng/ml unlabeled LT (B), 0.5 ng/ml biotinylated LT (C) or 0.5 ng/ml HRP-labeled LT (C). The cell morphology was observed after 16 hours of incubation.

FIG. 4: Binding of labeled-heat labile toxin to isolates HLT-L7M1, HLT-L7M2, HLT-L7M3, HLT-4N and HLT-29. The binding of biotinylated heat labile toxin on coated HLT-L7M1, HLT-L7M2, HLT-L7M3 was analyzed by ELISA. A) Bacterial solutions adjusted to indicated absorbance were coated and incubated with (+HLT) or without (−HLT) biotinylated heat labile toxin (100 ng/ml). B) Bacterial solutions adjusted to absorbance 1 were coated and treated (+PJ) or not (−PJ) with periodate prior to incubation with (+HLT) or without (−HLT) biotinylated heat labile toxin (100 ng/ml). Control=Lactobacillus acidophilus (DSM 9126).

C) The binding of HRP-heat labile toxin subunit B (100 ng/ml) on coated bacteria HLT-L7M1, HLT-4N and HLT-29 was analyzed by ELISA. Bacteria solutions adjusted to indicated absorbance were coated and treated (+PJ) or not (−PJ) with periodate prior to incubation with HRP-labeled LT (100 ng/ml). Control=Lactobacillus acidophilus (DSM 9126). Non-specific binding to HRP was controlled by incubation with free HRP (200 ng/ml) or another HRP-labeled Protein (200 ng/ml).

FIG. 5: Binding of HRP-labelled Cholera Toxin Subunit B to GM1.

The binding HRP-labelled cholera toxin subunit B (CTB) towells previously coated with GM1 and pre-treated (+PJ) or not (−PJ) with periodate was analyzed by ELISA. Unspecific binding of HRP-labelled toxin was revealed on wells coated with PBS.

FIG. 6: Binding of HRP-Cholera toxin subunit B to isolates CT-G45, CT-G51, CT-4K140, CT-6P24 and CT-92. The binding of HRP-cholera toxin subunit B on coated bacteria pre-treated or not with periodate was analyzed by ELISA. A) Bacterial solutions of the strain CT-G45, CT-G51, CT-4K142 and CT-6P24 adjusted to indicated absorbance were coated and incubated with (+CT) or without (−CT) HRP-labeled cholera toxin subunit B (20 ng/ml). B) Bacterial solutions adjusted to absorbance 1 were coated and treated (+PJ) or not (−PJ) with periodate prior to incubation with (+CT) or without (−CT) HRP-labeled cholera toxin subunit B (20 ng/ml). Control=Lactobacillus acidophilus (DSM 9126). C) Bacterial solution of the strain CT-92 adjusted to indicated absorbance was coated and incubated with (+CT) or without (−CT) HRP-labeled cholera toxin subunit B (20 ng/ml). D) Bacterial solutions of the strain CT-92 and CT-4K142 adjusted to indicated absorbance were coated and treated (+PJ) or not (−PJ) with periodate prior to incubation with HRP-labeled cholera toxin subunit B (20 ng/ml). Control=CT-non-binding E. coli strain. Non-specific binding to HRP was controlled by incubation with free HRP (200 ng/ml) or another HRP-labeled Protein (200 ng/ml).

FIG. 7: Inhibition of binding of HRP-cholera toxin subunit B to HT-29 cells

Fixed differentiated HT-29 cells were coated at 1×10⁵ cells/well and incubated for 2 hours at 37° C. with HRP-cholera toxin subunit B (10 ng/ml) in presence or not of 1×10⁸ cells/ml of the isolate CT-92 or a CT-non-Binding control strain (Negative control).

FIG. 8: Binding of HRP-labelled Shiga toxin 1 and 2 to Gb3.

The binding of HRP-labelled Shiga toxin 1 (0 to 200 ng/ml) to wells previously coated with Gb3 and pre-treated or not with periodate was analyzed by ELISA. Unspecific binding of HRP-labelled toxin was revealed on wells coated with PBS.

FIG. 9: Cytotoxic effect of Shiga toxins and HRP-labeled Shiga toxins on proliferating HT-29 cells.

HT-29 cells seeded at 5×10³ cells/well were cultured for 2 days. Culture medium was changed and added with 20μl of PBS containing indicated concentration of Shiga Toxin 1 (A), HRP-labeled Shiga Toxin 1 (B), Shiga Toxin 2(C) or HRP-labeled Shiga Toxin 2 (D). Survival rate was determined after 30 hours by mean of an MTT assay. Result of triplicates with standard deviation are presented.

FIG. 10: Binding of HRP-Shiga toxin 1 to isolates Stx1-P16B7, Stx1-P20F4, Stx1-P21A5 and Stx1-P21E8. The binding of HRP-Shiga toxin 1 (200 ng/ml) on coated bacteria pre-treated or not with periodate was analyzed by ELISA. Bacteria solutions adjusted to absorbance of 1 were coated. Control=Stx1 non-binding strain. Non-specific binding to HRP was controlled by incubation with free HRP (200 ng/ml) or another HRP-labeled Protein (200 ng/ml).

FIG. 11: Binding of HRP-Shiga toxin 2 to isolates STx2-10D1, Stx2-10-D7 and Stx2-15-A3. The binding of HRP-Shiga toxin 2 (200 ng/ml) and His-tagged Shiga toxin 2 subunit B on coated bacteria was analyzed by ELISA. The strains STx2-10D1, Stx2-10-D7 and Stx2-15-A3 were coated, incubated with His-tagged Shiga toxin 2 subunit B and with Rabbit anti-His antibody (A) or Mouse anti-His Mab (B). The strains STx2-10D1, Stx2-10-D7 and Stx2-15-A3 were coated and incubated with HRP-labeled Shiga toxin 2 (C). Control=Stx2 non-binding strain. Non-specific binding to HRP was controlled by incubation with free HRP (200 ng/ml) or another HRP-labeled Protein (200 ng/ml).

### DESCRIPTION OF THE INVENTION

A first aspect of the present invention is a method for isolating a naturally-occurring microorganism that displays a structure (A) on its surface, said structure being capable of binding a toxin from a pathogenic microorganism, comprising:
(a) bringing a composition comprising one or more microorganisms into contact with (i) said toxin and/or (ii) a binding moiety being capable of binding said structure (A); and
(b) obtaining one or more microorganisms bound by said toxin and/or binding moiety.

A second aspect of the present invention is a method for isolating a naturally-occurring microorganism that displays a structure (B) on its surface being capable of binding a surface receptor of a mammalian cell for a toxin from a pathogenic microorganism, comprising:
(a) bringing a composition comprising one or more microorganisms into contact with (iii) said receptor and/or (iv) a binding moiety being capable of binding said structure (B); and
(b) obtaining one or more microorganisms bound by said receptor and/or binding moiety.

Without being bound by theory, it is assumed that naturally-occurring microorganisms are capable of binding a toxin from another microorganism such as a pathogenic microorganism by way of a structure, herein called “structure (A)”.

Also, without being bound by theory, it is assumed that naturally-occurring microorganisms are capable of binding a surface receptor of a mammalian cell for a toxin from another microorganism such as a pathogenic microorganism by way of a structure, herein called “structure (B)”.

Structure (A) is believed to exert a function that is identical to or resembles the function of a receptor for such a toxin (sometimes referred to herein as (“toxin receptor”) on the surface of mammalian cells such as epithelial cells which, for example, are lined-up in the gut. This function may include a binding function. Thereby, structure (A) may be able to mimic the toxin receptor on the surface of mammalian cells. Yet, it may also be possible that structure (A) has a structure that resembles or mimics the toxin receptor, thereby it is believed that it may also be able to mimic the toxin receptor on the surface of mammalian cells.

These toxin receptors bind toxins from microorganism, which binding usually constitutes the onset of a disease caused by the microorganism secreting or having bound on its surface a toxin.

Without being bound by theory, structure (B) may, for example, bind to the surface receptor of a mammalian cell for a toxin, thereby occupying or masking the receptor such that a toxin from a microorganism can ideally not bind to said receptor or may be prevented from binding thereto.

The present inventors aimed at interfering with the binding of the toxin to a toxin receptor and, thus, developed the concept of the present invention, namely, providing naturally-occurring microorganisms which can bind a toxin from a microorganism such as a pathogenic microorganism and/or can bind a surface receptor of a mammalian cell for a toxin from a microorganism such as a pathogenic microorganism, thereby interfering with the toxin-toxin receptor interaction.

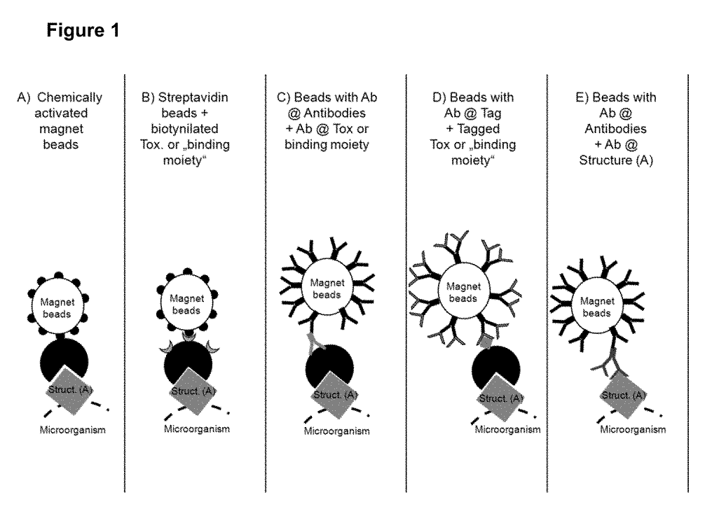

By way of example, in order to perform the method of the first aspect of the present invention, the skilled person can use a toxin from a pathogenic microorganism as “bait” in order to identify a structure (A). For example, in order to detect microorganisms (that display a structure (A)) bound to said toxin, the toxin may be labeled with a detectable marker or coupled to beads, such as magnetic beads and brought into contact with a composition comprising one or more microorganisms. Such an approach is described in the appended Example and illustrated in FIG. 1.

Without being bound by theory, the skilled person can prepare a recognition molecule against a structure (A), for example, against a glycosyl structures (carbohydrate structure) set out in Table 1 of U.S. Pat. No. 6,833,130 by means and methods known in the art. These structures are known to be bound by toxins from pathogenic microorganisms, in particular pathogenic bacteria. U.S. Pat. No. 6,833,130 describes such glycosyl structure.

Accordingly, it can be reasonably assumed that a recognition molecule which binds such a glycosyl structure may also recognize a structure (A) of a microorganism as described herein, because such a recognition molecule may be cross-reactive with a structure (A), since structure (A) may resemble or be homologous in terms of composition and/or conformation to a glycosyl structure being present on the surface of a mammalian cell that acts as toxin receptor. Hence, a binding moiety against a glycosyl structure known to be capable of binding a toxin from a pathogenic microorganism may also recognize a structure (A).

In order to check whether such a binding moiety can bind a microorganism that displays on its surface a structure (A), the skilled person can set up a competition assay by bringing both the toxin and the binding moiety into contact with a microorganism that displays a structure (A) on its surface. Provided that the binding moiety competes with the toxin on the binding, it is reasonable to assume that the binding moiety is directed against a structure (A), since it would otherwise not compete with the toxin.

In a preferred embodiment, in step (a) of the method of the first and/or second aspect of the present invention with the aim of reducing non-specific binding of microorganisms, in particular bacteria comprised in the composition, to the toxin preferably pasteurized bacteria, for example, Bifidobacterium ssp., may be added.

As mentioned above, a second aspect of the present invention is a method for isolating a naturally-occurring microorganism that displays a structure (B) on its surface being capable of binding a surface receptor of a mammalian cell for a toxin from a pathogenic microorganism, comprising:
(a) bringing a composition comprising one or more microorganisms into contact with (iii) said receptor and/or (iv) a binding moiety being capable of binding said structure (B); and
(b) obtaining one or more microorganisms bound by said receptor and/or binding moiety.

By way of example, in order to perform the above method, the skilled person can use as a “bait” for isolating a microorganism that displays a structure (B), a glycosyl structure (carbohydrate structure) known to be involved in the binding of a toxin (i.e., acting as a receptor for a toxin from a pathogenic microorganism). Such glycosyl structures are exemplified in Table 1 of U.S. Pat. No. 6,833,130.

Another “bait” that may be used is the glycosyl structure globotriasyl ceramide, Gb3, or Gb4. Gb3 and Gb4 are known to be bound by Stx1 and Stx2, respectively. For example, a glycosyl structure may be hooked up to a carrier such as a protein. It may be labeled with a detectable marker or coupled to beads such as magnetic beads which allow the isolation of a structure (B) bound by said gylcosyl structure.

In order to check whether such a glycosyl structure can bind a structure (B), the skilled person can set up a competition assay by bringing both the toxin and the glycosyl structure bound by said toxin into contact with a microorganism that displays a structure (B) on its surface. Provided that the microorganism competes with the toxin on the binding to said glycosyl structure, it is reasonable to assume that the microorganism displays a structure (B), since it would otherwise not compete with the toxin on the binding to said glycosyl structure.

In the alternative, a skilled person can use as a “bait” for isolating a microorganism that displays a structure (B), GUCY2C (guanylate cyclase 2C (heat stable enterotoxin receptor)), Cholera Toxin-GM1 Ganglioside Receptor, Clostridium perfringens enterotoxin-receptors Claudin-3 and Claudin-4. These “baits” are known receptors for a toxin from a pathogenic microorganism.

Once a structure (B) on the surface of a microorganism has been isolated, it can be used in the production of a binding moiety that allows the isolation of another structure (B), since another structure (B) may resemble the structure (B) identified as described above by using a glycosyl structure that acts as receptor for a toxin of a pathogenic microorganism. Alternatively, an anti-idiotypic antibody against an antibody directed against a glycosyl structure as described before may be generated that can be used as binding moiety that recognizes a structure (B).

The present invention provides a method that allows direct and selective isolation of naturally-occurring microorganisms that bind toxins from microorganisms and/or the corresponding toxin receptors present on mammalian cells. “Specific” means that the present invention provides a method that allows selective isolation of particular substrains of microorganisms that display a structure (A) or (B) on the surface. By way of example, the present invention may be used to selectively isolate from a composition comprising bacteria of a particular strain, substrains that display a structure (A) or (B) on the surface. Accordingly, the method of the present invention is not limited to the isolation of a particular class, order, family or strain of microorganisms.

Moreover, the present invention provides a method that allows direct isolation of microorganisms that display a structure (A) or (B) on the surface from a composition of mixed microorganisms. “Direct” means that separating the microorganism (displaying a structure (A) or (B) on its surfaces) and identifying the microorganism (as a microorganism that displays a structure (A) or (B) on its surface) are carried out in one procedural step. Accordingly, within the method of the present invention the obtained microorganism is not first isolated, e.g. by immuno-magnetic separation, and subsequently tested for the presence of a structure (A) or (B) on the surface, e.g. by use of PCR techniques or by assaying its capability to bind toxins and/or the corresponding toxin receptors present on mammalian cells.

The term “structure” when used herein means a structure on the surface of a microorganism, i.e., a microorganism displays a structure on its surface such that it gets or is in contact with the environment surrounding the microorganism. The term “structure” includes “structure (A)” and “structure (B)” as described herein.

The term “surface” herein includes any localization of structure (A) or structure (B) accessible from the outside of the microorganism. Accordingly, said structure may be directly exposed to the environment, i.e., be in direct contact with the environment, or may be in indirect contact, i.e., be in indirect contact with the environment, e.g. via pores (e.g. porins) that connect the periplasm with the environment. Hence, said structure may be present or integrated in the inner membrane, outer membrane, cell wall, pilus, flagellum or fimbria of a microorganism.

The exposure of the structure on the surface of a microorganism can result from natural expression, from processing done by the microorganism or by man either during the production process (e.g. enzymatic processing) or after ingestion by a mammal, such as a human or animal (e.g. modification through digestion enzymes).

Said structure may preferably be a peptide, protein, glycoprotein, lipid, glycolipid and/or carbohydrate structure. Said structure may also be a complex composed of one or more of a protein, glycoprotein, lipid, glycolipid and carbohydrate structure. Preferably, said structure is a carbohydrate structure-(sometimes also referred to herein as “carbohydrate molecule”). Preferably, said structure is a monosaccharide, disaccharide, oligosaccharide, polysaccharide, peptidaminoglycan, proteoglycan, glycoprotein, glycopeptides, lipopolysaccharides.

“Structure (A)” refers herein to a naturally-occurring structure capable of binding a toxin from a pathogenic microorganism. In preferred embodiments structure (A) competes with a toxin receptor of a mammalian cell, such as an animal or human cells for binding said toxin. Thereby structure (A) can reduce or impair the interaction between the toxin and its receptor. Moreover, structure (A) preferably captures toxins from a pathogenic microorganism in an environment.

“Structure (B)” refers herein to a naturally-occurring binding structure capable of binding a surface receptor of a mammalian cell for a toxin from a pathogen. In preferred embodiments structure (B) competes with said toxin for binding a toxin receptor of a mammalian cell, such as a cell from an animal or human cell. Preferably structure (B) thereby reduces or impairs the interaction between the toxin and its receptor.

The term “mammalian” when used herein refers to a mammal such as human and an animal, such as a dog, cat, cattle, pig, horse, camel, sheep, mouse, rat, poultry, fish preferably human. A “mammalian” cell is a cell from a mammal, preferably an epithelial or mucosal cell.

In a preferred embodiment the naturally-occurring microorganism that displays a structure A and/or structure B on its surface is a bacterium, yeast or fungus as described herein.

As used herein the term “receptor” or “toxin receptor” refers to a toxin-binding molecule present on the surface of a cell, preferably of a mammalian cell. Cells of interest include, for example epithelial or endothelial cells, in particular those that are part of a mammalian mucosal membrane, such as human or animal mucosal membranes. Toxins for which receptors are described include but are not limited to Shiga toxin Stx1, Stx2, Stx2c, Stx2d, Stx2e, Stx2f, C. difficile toxin A, C. difficile toxin B, C. botulinum toxin, Vibrio cholera toxin, E. coli heat labile enterotoxin Type 1, Escherichia coli heat-stable enterotoxin, Clostridium perfringens enterotoxin. Such toxin receptors include, but are not limited to, GUCY2C (guanylate cyclase 2C (heat stable enterotoxin receptor)), Heat labile enterotoxin- and Cholera Toxin-GM1 Ganglioside Receptor, Clostridium perfringens enterotoxin-receptors Claudin-3 and Claudin-4, Clostridium difficile A and B-receptors Combined Repetitive OligoPeptides (CROP's), Shiga toxin Stx1, Stx2, Stx2c, Stx2d Glycolipid receptor Globotriaosyl ceramide (Gb₃) or Shiga toxin Stx2e Glycolipid receptor Globotetraosyl ceramide (Gb4]).

The terms “bound by said toxin and/or binding moiety” and “bound by said receptor and/or binding moiety” as used herein include the meaning of “having bound said toxin and/or binding moiety” or “having bound said receptor and/or binding moiety”.

The “binding moiety” may be any recognition molecule that is capable of binding a structure as described herein, in particular, structure (A) or structure (B). A recognition molecule may provide the scaffold for one or more binding domains so that said binding domains can bind/interact with structure (A). However, a binding domain does not necessarily have to provide a scaffold for a binding domain, since a binding domain may also exist without the scaffold of the recognition molecule, for example, when the binding domain is a carbohydrate domain as explained below.

A scaffold could, for example, be provided by protein A, in particular, the Z-domain thereof (affibodies), ImmE7 (immunity proteins), BPTI/APPI (Kunitz domains), Ras-binding protein AF-6 (PDZ-domains), charybdotoxin (Scorpion toxin), CTLA-4, Min-23 (knottins), lipocalins (anticalins), neokarzinostatin, a fibronectin domain, an ankyrin consensus repeat domain or thioredoxin (Skerra, Curr. Opin. Biotechnol. 18, 295-304 (2007); Hosse et al., Protein Sci. 15, 14-27 (2006); Nicaise et al., Protein Sci. 13, 1882-1891 (2004); Nygren and Uhlen, Curr. Opin. Struc. Biol. 7, 463-469 (1997)).

The term “binding domain” characterizes in connection with the present invention a domain, preferably a protein domain or a carbohydrate domain, i.e., a molecule composed of one or more carbohydrates that are covalently linked via glycosidic bonds, or a combination of a protein domain and carbohydrate domain, i.e., the carbohydrate domain is covalently bound to the protein domain, which specifically binds/interacts with a given target epitope on structure (A) or structure (B). An “epitope” is antigenic and thus the term epitope is sometimes also referred to herein as “antigenic structure” or “antigenic determinant” of structure (A) or structure (B). The epitope of structure (A) or structure (B), respectively, can be a stretch of amino acids of a polypeptide/protein that represents a linear or non-linear epitope. However, the epitope can also be one or more carbohydrate residues of a carbohydrate molecule. A carbohydrate molecule can thus be composed of one carbohydrate residue that, for example, may be covalently bound to another molecule such as a protein, lipid, etc. However, a carbohydrate molecule can also be composed of more than one carbohydrate residues, e.g., it can be a complex carbohydrate molecule whose carbohydrate residues are connected to each other via glycosidic bonds. Such a complex carbohydrate molecule may be covalently bound to another molecule such as a protein, lipid, etc.

A binding domain when used herein is involved in antigen binding and, thus, can be an “antigen-interaction-site”. The term “antigen-interaction-site” defines, in accordance with the present invention, a motif of or within a binding domain, which is able to specifically interact with a specific antigen or a specific group of antigens, e.g. the identical antigen in different species. Said binding/interaction is also understood to define a “specific recognition”.

The term “specifically recognizing” (can be equally used with the term “directed to” or “reacting with”) means in accordance with this invention that the recognition molecule is capable of specifically interacting with and/or binding to at least two, preferably at least three, more preferably at least four amino acids of an epitope as defined herein, if the epitope is a stretch of amino acids of a polypeptide as defined herein. Said term also includes that the recognition molecule is capable of specifically interacting with and/or binding to one or more carbohydrate residues of a carbohydrate molecule as defined herein. “Specific” means that the recognition molecule recognizes (or is directed to or reacts with) structure (A) or structure (B) A preferred binding moiety is an antibody, or a lipocalin. Another preferred binding moiety is a carbohydrate domain as described above. A further preferred binding moiety is a lectin.

The term “antibody” also includes but is not limited to polyclonal, monoclonal, monospecific, polyspecific such as bispecific, non-specific, humanized, human, single-chain, chimeric, synthetic, recombinant, hybrid, mutated, grafted, and in vitro generated antibodies, with a monoclonal antibody being preferred. Said term also includes domain antibodies (dAbs) and nanobodies as well as fragments such as scFvs, diabodies, tribodies, of an antibody. Also included are antibody-fusion proteins, e.g. whereby an antibody is fused to a further protein serving, e.g. as a tag or label, etc.

A “lipocalin” is preferably selected from the group consisting of muteins of retinol-binding protein (RBP), bilin-binding protein (BBP), apolipoprotein D (APO D), neutrophil gelatinase associated lipocalin (NGAL), tear lipocalin (TLPC), α2-microglobulin-related protein (A2m), 24p3/uterocalin (24p3), von Ebners gland protein 1 (VEGP 1), von Ebners gland protein 2 (VEGP 2), and Major allergen Can f1 precursor (ALL-1). In related embodiments, the lipocalin mutein is selected from the group consisting of human neutrophil gelatinase associated lipocalin (hNGAL), human tear lipocalin (hTLPC), human apolipoprotein D (APO D) and the bilin-binding protein of Pieris brassicae. Lipocalins are described, for example, in WO 2011/069992 or WO 2008/015239.

In some embodiments of the present invention the toxin and/or a binding moiety is coupled to a label, tag, antibody and/or bead. Labels and tags for biological molecules as well as methods to equip biological molecules with a tag or label are known to a person skilled in the art.

In further embodiments of the present invention the antibody, lectin or lipocalin is coupled to a bead. As use herein “coupled” includes any direct or indirect coupling of molecules. An indirect coupling thereby refers to a connection via one or more linking molecules. Such linking molecules can be chemical linkers, labels, tags, antibodies or beads.

Within the present invention “toxin” includes toxins in their naturally occurring form, inactivated toxins and fragments or derivatives of toxins such as recombinant toxins of a pathogenic microorganism, for example pathogenic microorganisms. Toxins in connection with present invention are preferably toxins which are relevant for endangering health and/or well-being of humans or non-human animals, such cattle, pig, horse, sheep, goat, cats, dogs, ducks, goose, chicken, fish, etc. A toxin is preferably a toxin produced either by a bacterium belonging to a family selected from the group consisting of Enterobacteriaceae, Clostridiaceae, Vibrionaceae, Staphylococcaceae, Streptococcaceae, Helicobacteraceae, Pseudomonadaceae, Pasteurellaceae, Chlamydiaceae, Campylobacteraceae, Aeromonadaceae, Neisseriaceae, Listeriaceae, Corynebacteriaceae, Aeromonadales, Bacteroidaceae, Bordetella, Bacillaceae or a protozoa belonging to a family selected from the group consisting of Acanthamoebidae, Amoebida, Hexamitidae, Cryptosporidiidae or a fungi belonging to a family selected from the group consisting of Saccharomycetaceae, Trichocomaceae, Clavicipitaceae, Nectriaceae.

Furthermore “toxin” refers herein preferably to a toxin produced either by a bacterium belonging to a genus selected from the group consisting of Enterobacter, Echerischia, Shigella, Clostridium, Vibrio, Staphylococcus, Streptococcus, Helicobacter, Pseudomonas, Haemophilus, Chlamydia, Campylobacter, Salmonella, Citrobacter, Yersinia, Pasteurella, Neisseria, Listeria, Corynebacterium, Klebsiella, Aeromonas, Serratia, Proteus, Bacteroides, Bordetella, Bacillus or a protozoa belonging to a genus selected from the group consisting of Acanthamoeba, Entamoeba, Giardia, Cryptosporidium or a fungi belonging to a genus selected from the group consisting of Candida, Penicillium, Aspergillus, Claviceps, Paecilomyces, Fusarium. Furthermore toxin includes toxins made in the gut. Preferably “toxin” refers to an enterotoxin produced by a pathogenic microorganism.

Within the context of the present invention the term “toxin” includes, but is not limited to toxins of the following list:

E. coli: Heat labile toxin (LT), Heat stabile toxin (ST), Verotoxins/Shiga like toxins (Stxs), Cytotoxins, endotoxins (LPS), EnteroAggregative ST toxin (EAST),

Shigella: Shiga toxin (STxs), Shigella enterotoxins 1 (ShET1), Shigella enterotoxins 2 (ShET2), Neurotoxin;

Salmonella: Cytolethal distending toxins (Cdt), AvrA toxin;

Yersinia: Cytotoxic necrotizing facto (CNFy), Yersinia murine toxin (Ymt), Yst toxin, Toxin complex (TCa), Heat stabile toxin;

Enterobacter: E. cloacae leukotoxin, Shiga-like toxin II,

Klebsiella: heat-stable like enterotoxins, extracellular toxic complex (ETC);

Serratia: Hemolysins (Shl), Pore-forming Toxin (PFT),

Proteus: α-hemolysin (HlyA),

Citrobacter: heat-stable like toxin, Cytotoxins;

Clostridium: C. perfringens alpha-toxin (CpPLC), C. perfringens beta toxin, C. perfringens enterotoxin (CPE), C. difficile enterotoxins (Tcd), C. butulinum Neurotoxins, C. tetani Tetanospasmin, C. butulinum C2 toxin, C. butulinum C3 toxin, C. perfringens epsilon-toxin (s-toxin), C. perfringens iota-toxin (t-toxin), tetanus neurotoxin (TeNT), theta-toxin/PFO (perfringolysin O), C. spiroforme (spiroforme toxin), C. septicum (a-toxin), Lecithinase;

Vibrio: Cholera toxins (CTx), accessory cholera enterotoxin (Ace), RTX toxin, zona occludens toxin (Zot), Cholix toxin;

Staphylococcus: α-hemolysin, δ-hemolysin, δ-hemolysin, γ-hemolysin, Exfoliative toxins (Exofoliatins), Panton-Valentine leukocidin (PVL), staphylococcal enterotoxins (SE), Toxic shock syndrome toxin-1 (TSST-1)

Streptococcus: β-haemolysin/cytolysin, CAMP factor, Streptolysin O, Streptolysin S, Pneumolysin, S. pyogenes Exotoxins (PSE),

Helicobacter: vacuolating cytotoxin A (VacA), Cytolytic toxins

Pseudomonas: Exotoxins (ex: ExoA, ExoS, ExoT, ExoU, ExoY), Phospholipase C (PLC)

Pasteurella: Pasteurella Multocida Toxin (PMT), RTX toxins

Bacillus: B. weihenstephanensis endotoxins, B. cereus Hemolysin BL (Hbl), B. cereus onhemolytic Enterotoxin (Nhe), B. cereus Cytotoxin K (CytK), B. cereus emetic toxin, B. cereus toxin (Cereolysin), B. anthracis (Anthrax toxin), B. thuringiensis δ-,endotoxins (Cry toxins),

Campylobacter: Cytolethal distending toxin (cdtA, cdtB, cdtC), cholera-like enterotoxin

Aeromonas: Aerolysin Cytotoxic Enterotoxin (ACT), ADP-ribosylation toxin, a-hemolysins, b-hemolysins, Heat labile toxin (LT+), Heat stabile toxin (ST+)

Neisseria: endotoxins (LPS)

Bordetella: B. pertussis (pertusis toxin), Adenylate cyclase toxin, Tracheal cytotoxin, Dermonecrotic (heat-labile) toxin, endotoxins (LPS)

Haemophilus: Endotoxin (LOS), Cytolethal distending toxins (HdCDT), Hemolysins

Chlamydia: Endotoxins

Corynebacteria: Cytotoxins, Diphteria toxin, Exotoxins

Bacteroides: Bacteroides fragilis toxin (bft)

Listeria: Listeriolysin O

In a preferred embodiment, the toxin is Heat labile toxin (LT), Heat stabile toxin (ST), Verotoxins/Shiga like toxins (Stxs), Cytotoxins, endotoxins (LPS), EnteroAggregative ST toxin (EAST), Shiga toxin (STxs), Shigella enterotoxins 1 (ShET1), Shigella enterotoxins 2 (ShET2), Neurotoxin, Cytolethal distending toxins (Cdt), AvrA toxin, Cytotoxic necrotizing facto (CNFy), Yersinia murine toxin (Ymt), Yst toxin, Toxin complex (TCa), Heat stabile toxin, E. cloacae leukotoxin, Shiga-like toxin II, heat-stable like enterotoxins, extracellular toxic complex (ETC), Hemolysins (Shl), Pore-forming Toxin (PFT), α-hemolysin (HlyA), heat-stable like toxin, Cytotoxins, C. perfringens alpha-toxin (CpPLC), C. perfringens beta toxin, C. perfringens enterotoxin (CPE), C. difficile enterotoxins (Tcd), C. butulinum Neurotoxins, C. tetani Tetanospasmin, C. butulinum C2 toxin, C. butulinum C3 toxin, C. perfringens epsilon-toxin (s-toxin), C. perfringens iota-toxin (t-toxin), tetanus neurotoxin (TeNT), theta-toxin/PFO (perfringolysin O), C. spiroforme (spiroforme toxin), C. septicum (a-toxin), Lecithinase, Cholera toxins (CTx), accessory cholera enterotoxin (Ace), RTX toxin, zona occludens toxin (Zot), Cholix toxin, α-hemolysin, β-hemolysin, δ-hemolysin, γ-hemolysin, Exfoliative toxins (Exofoliatins), Panton-Valentine leukocidin (PVL), staphylococcal enterotoxins (SE), Toxic shock syndrome toxin-1 (TSST-1), β-haemolysin/cytolysin, CAMP factor, Streptolysin O, Streptolysin S, Pneumolysin, S. pyogenes Exotoxins (PSE), vacuolating cytotoxin A (VacA), Cytolytic toxins, Exotoxins (ex: ExoA, ExoS, ExoT, ExoU, ExoY), Phospholipase C (PLC), Pasteurella Multocida Toxin (PMT), RTX toxins, B. weihenstephanensis endotoxins, B. cereus Hemolysin BL (Hbl), B. cereus, onhemolytic Enterotoxin (Nhe), B. cereus Cytotoxin K (CytK), B. cereus emetic toxin, B. cereus toxin (Cereolysin), B. anthracis (Anthrax toxin), B. thuringiensis δ-,endotoxins (Cry toxins), Cytolethal distending toxin (cdtA, cdtB, cdtC), cholera-like enterotoxin, Aerolysin Cytotoxic Enterotoxin (ACT), ADP-ribosylation toxin, a-hemolysins, b-hemolysins, Heat labile toxin (LT+), Heat stabile toxin (ST+), endotoxins (LPS), B. pertussis (pertusis toxin), Adenylate cyclase toxin, Tracheal cytotoxin, Dermonecrotic (heat-labile) toxin, endotoxins (LPS), Endotoxin (LOS), Cytolethal distending toxins (HdCDT), Hemolysins, Endotoxins, Cytotoxins, Diphteria toxin, Exotoxins, Bacteroides fragilis toxin (bft), Listeriolysin O, or rota virus toxin (NSP4).

Without limiting the invention, some specific examples of toxin producing bacteria and their toxins are described below.

Clostridium difficile

Clostridium difficile infection (CDI) or Clostridium difficile-associated diarrhea (CDAD) is widely accepted to be one of the leading causes of nosocomial infection with a recurrence rate that typically ranges from 5% to 20%. CDI related morbidity and mortality rate is outpacing both antibiotic-resistant staphylococcus (MRSA) and enterococcus (VRE) (20).

In the last decade, CDIs have become more frequent, more severe, more refractory to standard therapy, and more likely to relapse after initial treatment (21). This is attributed to the common use of broad-spectrum antibiotics and to a new hypervirulent strain of C. difficile, alternatively designated as BI, NAP1, or ribotype 027 toxinotype III (22).

Today CDIs are a major public health concern, accounting for significant morbidity and mortality, extended hospitalization, and high health-care expenses (23).

The primary virulence factors of C. difficile are two toxins, toxin A (TcdA) and toxin B (TcdB) (24). They belong to the family of large clostridial toxins (LCTs) (25). These lead to the glycosylation and thereby inactivate Rho proteins, which in turn leads to the destruction of the intestinal cells (26). To cause this toxic effect, the toxins must reach the inside of the cell. This is dependent on the binding to specific membrane receptors of the target cells (27).

The C-termini of TcdA and TcdB consist of highly repetitive structures termed combined repetitive oligopeptides (CROPs) that bind sugar moieties on the surface of host cells (28). TcdA has been reported to bind to the human I, X, and Y blood antigens as well as a human glycosphingolipid which all have a core p-Gal-(1,4)-3-GlcNAc structure (29). It is not known which of these, if any, serve as the native ligand in the human colon. No receptors have been described for TcdB.

The present invention provides a pharmaceutical composition containing at least one non-pathogenic microorganism naturally expressing a binding structure for the toxins TcdA and/or TcdB. Such microorganisms are specifically and strongly binding the said toxin(s). Such microorganisms can be used to displace the toxins from their natural receptor(s) present on the surface of intestinal cells in order to treat, cure, abate or prevent Clostridium difficile associated diseases.

Shiga Toxins

The most common sources for Shiga toxin are the bacteria Shigella dysenteriae and the Shigatoxigenic group of Escherichia coli (STEC). STEC accounts for an estimated 314,000 infections annually in industrialized countries, including approximately 110,000 people in the United States and 97,000 people in the European Union according to the Centers for Diseases Control and Prevention (CDC) and the (EMEA), respectively. Shigella also causes approximately 580,000 cases annually among travelers and military personnel from industrialized countries.

Symptom induced by STEC may be restricted to mild diarrhea but can evolve to hemorrhagic colitis, and potentially to life-threatening Hemolytic Uremic Syndrome (HUS). HUS is characterized by hemolytic anemia, thrombic thrombocytopenia, and renal failure. About 5% to 20% of STEC infected individuals may develop HUS (CDC). HUS presents a 5% to 10% fatality rate and survivors may have lasting kidney damage (30, 31, 32).

The most important virulence factors responsible for the evolution of the complications are the Shiga toxin Stx1 and Stx2. HUS is induced by the systemic action of Stx2 on the kidney (33). During the early stages of human infections, STEC may colonize the gut at high levels, exposing the host to sustained high concentrations of Stx and increasing the likelihood of systemic complications. As disease progresses, the numbers of STEC decrease markedly due to response of the immune system. However this response occurs too slowly to prevent Stx-induced complications.

Both Stx1 and Stx2 toxins bind to the eukaryotic carbohydrate receptor globotriaosyl ceramide (Gb₃) (or Gb₄ as is the case for Stx2e). Stx2 that binds preferentially to Gb3 variants in kidney tissue is associated with more severe disease outcome and is 100- to 400-fold more potent than Stx1 (33).

The present invention provides a pharmaceutical composition containing at least one microorganism naturally expressing a binding structure for the toxins Stx1 and/or Stx2. Such microorganisms are specifically and strongly binding the said toxin(s). Such microorganisms can be used to displace the toxins from their natural receptor(s) present on the surface of intestinal cells in order to treat, cure, abate or prevent Shiga toxin associated diseases and symptoms.

Travelers' Disease (ETEC)

Travelers' Diarrhea (TD) is the most common infectious disease to affect travelers from industrialized countries to developing countries with a reported incidence of 20% to 66% during the first two weeks in the country of destination. Enterotoxigenic Escherichia coli (ETEC) is the single most common cause of TD in adult travelers, being responsible for 20 to 40% of all TD cases worldwide (34, 35, 36). ETEC can be estimated to cause disease in up to 10 million travelers per year (CDC). Transmission of ETEC is usually from fecal contaminated food and water. The infection occurs 10 hours to 3 days after exposure typically causing profuse watery diarrhea sometimes with low grade fever, abdominal cramping and/or vomiting. Furthermore, there is growing evidence that acute illness experienced by these visitors to developing countries can lead to more long-term health conditions, ranging from functional gastrointestinal disorder, like irritable bowel syndrome, to reactive arthritis in approximately 10 percent of individuals recovering from an episode of travelers' diarrhea.

Responsible for the diarrhea are two enterotoxins produced by ETEC, a heat-stable (ST) and/or a heat-labile (LT) enterotoxin. Roughly one third of all ETEC strains isolated globally have been reported to be ST-only strains, one third ST/LT and one third LT-only strains (37). Furthermore, ETEC is colonizing the intestine through interaction between Colonization factors (CFs) on the bacterial surface and receptors present on the intestinal epithelium. Approximately 60% to 90% of ST/LT strains express CFA/I or CS1 to CS6, whereas these CFs are expressed by approximately 40% to 70% of ST-only strains and are very rarely expressed in LT-only strains. CS6 (alone or in combination with CS4 or CS5) was identified in 41% to 52% of all CF-positive strains making it the most common CF in these studies (36, 38, 39, 40, 41).

LT:

The natural cell surface receptor for LT is ganglioside GM1 (Gal-b1,3-GalNAcb1,4-(NeuAc-α2,3)-Gal-b1,4-Glc-b1,1-ceramide). The oligosaccharide part of GM1 (GM1-OS) is responsible for binding to LT (42).

STa:

The receptor for STa was shown to be the guanylyl cyclase-C (GC-C). This receptor is highly glycosylated, containing 8-10 W-linked glycosylation sites, depending on the species.

It is not clear whether the sugar residues are essential for ligand binding. Enzymatic deglycosylation of mature GC-C had no effect on binding affinity and activation. On the other side, in the same study, deglycosylation of GC-C by PNGase F resulted in a loss of STa binding (43).

CFs:

The colonization factor CS6 was demonstrated to have a high affinity for the sulfatide (S03-3Galb1Cer) (44). Also it was shown that colonization factor (CF) antigen I (CFA/I) binds to glycosphingolipids associated with blood group antigens, e.g., Le^{a} and that CS1 and CS4 present a similar glycosphingolipid binding pattern.

The present invention provides a pharmaceutical composition containing at least one microorganism naturally expressing a binding structure for the toxins LT and/or ST. Such microorganisms are specifically and strongly binding the said toxin(s). Such microorganisms can be used to displace the toxins from their natural receptor(s) present on the surface of intestinal cells in order to treat, cure, abate or prevent ETEC associated diseases and symptoms.

Cholera Toxin

Cholera is an acute, diarrheal illness caused by infection of the intestine with the bacterium Vibrio cholerae. An estimated 3 to 5 million cases and over 100,000 deaths occur each year around the world (45).

Heat labile toxin (LT) from ETEC and Cholera toxin are highly identical and both were reported to bind the same carbohydrate receptor GM1 described above. However, slightly different specificity were reported (46, 47).

The present invention provides a pharmaceutical composition containing at least one microorganism naturally expressing a binding structure for the Cholera toxins. Such microorganisms are specifically and strongly binding the said toxin. Such microorganisms can be used to displace the toxins from their natural receptor(s) present on the surface of intestinal cells in order to treat, cure, abate or prevent Vibrio cholerae associated diseases and symptoms.

Unless defined otherwise, the term “microorganism(s)” used herein includes bacteria, viruses, fungi (including unicellular and filamentous fungi), yeasts, protozoa and multi-cellular parasites. Typical sources of microorganisms described herein include faeces, gut, skin, nose, ear, mouth, eye, urogenital tract, breast milk, foods (including but not limited to: milk products, meet etc. . . . ), pure cultures, soil, water and plants.

That being so, “microorganism(s)” as applied in the methods, compositions and uses of the present invention may designate different microorganisms (dependent on the context), namely:
a) a microorganism comprised in the composition which is used in a method according to the present invention as source material,
b) a pathogenic microorganism which can be in the source material,
c) a pathogenic microorganism which has (i.e., expresses and secretes) a toxin, d) a microorganism that is obtainable or obtained by a method according to the present invention,
e) a naturally-occurring microorganism that displays a structure (A) and/or structure (B) on its surface.

To a): The term “microorganism” when used in the context of “a composition comprising one or more microorganisms” or “microorganism comprised in said composition” refers to bacteria, viruses, fungi (including unicellular and filamentous fungi), yeasts, protozoa and multi-cellular parasites. Such a microorganism is preferably non-pathogenic.

In preferred embodiments of the invention a microorganism comprised in the composition, which is used as source material, is a bacterium, virus, fungus (including unicellular and filamentous fungi), yeast, protozoon or a multi-cellular parasite. As said, such a microorganism is preferably non-pathogenic.

To b): A microorganism comprised in a composition which is used in a method according to the present invention as source material may, though it is less preferred, be pathogenic. If so, it can be made non-pathogenic by means and methods known in the art once it has been isolated in accordance with the methods of the present invention.

To c): In the present invention “pathogenic microorganisms”, which have (i.e., express and secrete) a toxin, include microorganisms as described herein that can cause lesion and/or disease of mucosa, including but not limited to buccal mucosa, esophageal mucosa, gastric mucosa, intestinal mucosa, nasal mucosa, olfactory mucosa, oral mucosa, bronchial mucosa, uterine mucosa, endometrium (mucosa of the uterus), vaginal mucosa, penile mucosa by, inter alia, a toxin. “Pathogenic microorganisms” further include microorganisms that cause lesions and/or disease of the gastrointestinal tract such as diarrhea.

Within this invention “pathogenic microorganisms” include, but are not limited to, the microorganisms mentioned herein, in particular those mentioned in the context of the term “toxin”. Non-limiting example are Clostridium difficile, Clostridium perfringens, Shiga toxigenic E. coli (STEC), Shigella dysenteriae, enterotoxigenic E. coli, Staphylococcus pneumonia, Staphylococcus aureus, Bacillus cereus, Chlamydia trachomatis, Acanthamoeba, Candida albicans, Helicobacter pylori, Pseudomonas spp., Aeromonas caviae, Aeromonas sobria und Aeromonas hydrophila, Entamoeba histolyticum, Porcine enterotoxigenic E. coli (ETEC), Vibrio cholera, Salmonella spp., Campylobacter spp., Yersinia enterocolitica, H. influenza, H. parainfluenza, Norovirus, Rotavirus, or Adenovirus.

To d) When “microorganism” is used herein to describe a microorganism obtainable or obtained by a method of the present invention, the term “microorganism” refers to bacteria, yeasts or fungi (includes unicellular and filamentous fungi).

Microorganisms obtained or obtainable by the present invention can be non-pathogenic, pathogenic, harmful, live, dead or killed, wherein “killed” means that the present invention comprises a step for killing microorganisms. Examples of methods of providing killed microorganism include, but are not limited to, treatment with chemical agents such as formalin, thiomersal, or streptomycin or other bactericidal antibiotic, or exposure to heat or UV irradiation.

However, preferably the obtained or obtainable microorganism is non-pathogenic. Non-pathogenic microorganisms preferably include, but are not limited to microorganisms categorized as Generally Recognized As Safe (GRAS). Non-pathogenic microorganisms further preferably include, but are not limited to lactic acid bacteria or bifidobacteria. They may also include opportunistic pathogen microorganisms.

To e) Aim of a method according to the present invention is to isolate a naturally-occurring microorganism that displays a structure (A) and/or structure (B) on its surface. The term “naturally-occurring microorganism” refers here to a microorganism that is present in nature and which is not genetically modified, genetically-engineered or made recombinant by man. For avoidance of doubt, “genetically-engineered” does not include subcloning. Also, the term “naturally-occurring microorganism” includes a microorganism that has been isolated from nature and selected for a specific property, trait, etc. without genetic modifications, but merely by, e.g., selection.

The terms “isolated” or “isolation” as used herein, refer to the separation of the obtained microorganism from the starting composition comprising a plurality of microorganisms, e.g. a sample of the natural environment of the microorganism or any other used source material. Within the present invention, a naturally-occurring microorganism, separated from some or all of the coexisting materials in the starting composition, is isolated. Such a microorganism could be part of a composition, and is still to be regarded as being isolated, provided that the composition does not correspond to its natural environment or any other used source material.

In some embodiments the present invention refers to a method comprising purifying the obtained microorganism, thereby obtaining a purified microorganism. The term “purified” does not require absolute purity; rather, it is intended as a relative definition. Microorganisms obtained by use of the present invention may be conventionally purified to microbiological homogeneity, i.e. they grow as single colonies when streaked out on agar plates by methods known in the art. Preferably, the agar plates that are used for this purpose are selective for the microorganism isolated by the methods of the present invention. Such selective agar plates are known in the art

In some preferred embodiments the present invention refers to a method comprising culturing the obtained microorganism. The term “culturing” includes cultivating said microorganism under conditions suitable for said microorganism to survive and/or reproduce and express the structure (A) or (B). Said term also includes increasing the amount and/or accessibility of structure (A) and/or (B) by culturing the composition and/or the obtained microorganisms at suitable conditions and/or media, preferable by using stress conditions. Stress condition may be, for example, high density cultivation, reduction of nutrients, increase of oxygen for anaerobic organisms, decrease of oxygen for aerobic microorganism, pH change, and others known to those skilled in the art.

Suitable conditions comprise inter alia aerobic and anaerobic culturing conditions. Preferably “culturing” as used herein refers to the culturing of bacteria or fungi obtained by the present invention. Methods for culturing bacteria or fungi are well known in the art.

In some embodiments the present invention refers to a method comprising testing the obtained microorganism for its capacity to neutralize the toxin and/or reducing the pathogenicity of a pathogenic microorganism.

In another aspect, the present invention provides a microorganism obtainable by a method according to the present invention. Preferably, such a microorganism is capable of binding a toxin from a pathogenic microorganism and/or capable of binding a surface receptor of a mammalian cell for a toxin from a pathogenic microorganism.

Another aspect of the present invention is an analog, variant or fragment of a microorganism obtainable by a method according to the present invention, which is preferably capable of binding a toxin from a pathogenic microorganism and/or capable of binding a surface receptor of a mammalian cell for a toxin from a pathogenic microorganism.

According to the present invention the term “analog” includes a dead or inactivated microorganism of the present invention, preferably of a microorganism that displays a structure (A) or structure (B) on its surface.

According to the present invention the term “variant” includes mutants of a microorganism and microorganisms related to microorganisms of the present invention, wherein said mutants or related microorganisms display a structure (A) or structure (B) on the surface. Preferably, “mutant” refers to a microorganism of the present invention, which harbours naturally-occurring, spontaneous mutations in the genome.

A “fragment” of a microorganism encompasses any part of the cells of a microorganism of the present invention. Preferably, said fragment is a membrane fraction obtained by a membrane-preparation. Membrane preparations of microorganisms can be obtained by methods known in the art, for example, by employing the method described in Rollan et al., Int. J. Food Microbiol. 70 (2001), 303-307, Matsuguchi et al., Clin. Diagn. Lab. Immunol. 10 (2003), 259-266 or Stentz et al., Appl. Environ. Microbiol. 66 (2000), 4272-4278 or Varmanen et al., J. Bacteriology 182 (2000), 146-154. Alternatively, a whole cell preparation is also envisaged. Preferably, the herein described fragment of a microorganism of the present invention comprises a structure (A) or structure (B).

Within this invention terms like “analog, mutant or fragment thereof” include the meaning “analog thereof, mutant thereof or fragment thereof”. Furthermore “analog, mutant or fragment thereof” includes combinations of an analog, mutant and or fragment. For example, a fragment of an analog of a microorganism according to the present invention

A microorganism of the present invention or an analog, variant or fragment thereof might be inactivated, lyophilized, spray-dryed or dried, wherein said microorganism, analog variant or fragment preferably retains its capability of binding a toxin from a pathogenic microorganism and/or a surface receptor of a mammalian cell for a toxin from a pathogenic microorganism.

In some preferred embodiments the microorganism obtainable by a method according to the present invention, which is capable of binding a toxin from a pathogenic microorganism and/or capable of binding a surface receptor of a mammalian cell for a toxin from a pathogenic microorganism, or the analog, variant or fragment thereof, is capable of neutralizing the toxin and/or reducing the pathogenic effect of a pathogenic microorganism. In other preferred embodiments said microorganism, analog, variant or fragment is in the form of a lysate or fraction.

According to the present invention the term “lysate” means a solution or suspension in an aqueous medium of cells of a microorganism of the present invention that are broken. However, the term should not be construed in any limiting way. The cell lysate comprises, e.g., macromolecules, like DNA, RNA, proteins, peptides, carbohydrates, lipids and the like and/or micromolecules, like amino acids, sugars, lipid acids and the like, or fractions of it. Additionally, said lysate comprises cell debris which may be of smooth or granular structure. Methods for preparing cell lysates of microorganism are known in the art, for example, by employing French press, cells mill using glass or iron beads or enzymatic cell lysis and the like. In addition, lysing cells relates to various methods known in the art for opening/destroying cells. The method for lysing a cell is not important and any method that can achieve lysis of the cells of a microorganism of the present invention may be employed. An appropriate one can be chosen by the person skilled in the art, e.g. opening/destruction of cells can be done enzymatically, chemically or physically.

According to the invention, lysates can also be preparations of fractions comprising a microorganism of the present invention. “Fractions” as used herein can be obtained by methods known to those skilled in the art, e.g., chromatography, including, e.g., affinity chromatography, ion-exchange chromatography, size-exclusion chromatography, reversed phase-chromatography, and chromatography with other chromatographic material in column or batch methods, other fractionation methods, e.g., filtration methods, e.g., ultrafiltration, dialysis, dialysis and concentration with size-exclusion in centrifugation, centrifugation in density-gradients or step matrices, precipitation, e.g., affinity precipitations, salting-in or salting-out (ammoniumsulfate-precipitation), alcoholic precipitations or other protein chemical, molecular biological, biochemical, immunological, chemical or physical methods to separate components a lysate.

In some embodiments the present invention refers to a method comprising admixing the isolated microorganism with a pharmaceutically acceptable carrier. “Isolated microorganism” refers here to a naturally-occurring microorganism that displays a structure (A) or structure (B) on its surface.

The term “pharmaceutically acceptable” means approved by a regulatory agency or other generally recognized pharmacopoeia for use in animals, and more particularly in humans.

The term “carrier” refers to a diluent, adjuvant, excipient, or vehicle with which a substance is administered. A carrier is pharmaceutically acceptable, i.e. if it is non-toxic to a recipient at the dosage and concentration employed. A pharmaceutically acceptable carrier is preferably isotonic, hypotonic or weakly hypertonic and has a relatively low ionic strength. Pharmaceutical carriers can be sterile. Pharmaceutical carriers for various applications are well known for a person skilled in the art and described in the professional literature. Pharmaceutical carriers are e.g. described in “Remington's Pharmaceutical Sciences” by E. W. Martin or in “Handbook of Pharmaceutical Excipients” by R. C. Rowe et al. The term “carrier” is herein interchangeable with the term “excipient” and vice versa.

The invention provides pharmaceutical preparations for administration of a microorganism obtainable according to the invention or an analog, variant or fragment thereof. These preparations include one or several of said microorganisms, analogs, variants or fragments, or lysates or fraction thereof, in appropriate quantity within an acceptable pharmaceutical excipient. The invention also provides methods of administration to reach appropriate efficacy.

The term “composition” is used herein in two modes.

Firstly, “composition” designates the source material of methods of the present invention. This includes the term “bringing a composition comprising one or more microorganisms into contact with”. In some embodiments said composition comprises one or more microorganisms that are comprised in human or animal faeces. In preferred embodiments said composition is from a human sample, animal sample, soil, water, food or culture of microorganisms.

Within the present invention human or animal samples preferably refers to body fluids (e.g. blood, urine, ichor, secretions), mucus, skin, tissue or faeces.

Secondly, “composition” designates compositions that can be obtained by (or are obtainable by) a method according to the present invention.

One aspect of the present invention is a composition comprising a microorganism, preferably obtainable by a method according to the present invention, which is preferably capable of binding a toxin from a pathogenic microorganism and/or capable of binding a surface receptor of a mammalian cell for a toxin from a pathogenic microorganism, or a composition comprising the analog, variant or fragment of said obtainable microorganism, wherein said analog, fragment, variant or obtainable microorganism, is preferably capable of neutralizing a toxin and/or reducing the pathogenic effect of a pathogenic microorganism. Said aspect of the present invention also refers to a composition wherein the above defined analog, variant, fragment or obtainable microorganism is in the form of a lysate or fraction.

In a preferred embodiment the present invention corresponds to a composition as defined in the preceding paragraph, which is a pharmaceutical composition, preferably for use in the nutrition of non-human animals.

In one aspect the present invention is a pharmaceutical composition as defined in the preceding paragraph or a microorganism obtainable by a method according to the present invention, which is preferably capable of binding a toxin from a pathogenic microorganism and/or capable of binding a surface receptor of a mammalian cell for a toxin from a pathogenic microorganism and further preferably capable of neutralizing a toxin and/or reducing the pathogenic effect of a pathogenic microorganism; or an analog, variant or fragment thereof; or a lysate or fraction of said analog, variant, fragment or obtainable microorganism; for use in a method of treating, alleviating, or preventing a gastrointestinal disease.

As used herein “gastrointestinal disease” includes infections of the gastro-intestinal system, chronic diseases of the gastro-intestinal system that are associated with enterotoxin-producing microorganisms.

In preferred embodiments the present invention is a composition for the use in a method of treating, alleviating, or preventing a gastrointestinal disease, wherein said gastrointestinal disease is a gastrointestinal infection. Preferably, said gastrointestinal infection is caused by a bacterium, yeast, fungus, virus, or protozoan organism. Such microorganisms can either initially cause a gastrointestinal disease or occur in consequence of another gastrointestinal disease.

In a preferred embodiment the present invention is a composition for the use in a method of treating, alleviating, or preventing a gastrointestinal disease as defined above, which is administered by enteral application. Pharmaceutical compositions of the present invention may be administered by enteral application.

“Enteral application” herein includes the following administrations: oral, peroral, gastric, intragastral, topic, nasal and rectal. “Gastric” application involves the use of a tube through the nasal passage or a tube in the belly leading directly to the stomach.

Preferred administration forms include, but are not limited to pill, tablet, capsule, caplet, powder, lyophilisate, spray-dried composition, granule, pellets, liquid, solution, suspension, emulsion, gel, suppository, enema or rectal infusion. A composition of the present invention or a processing product thereof can be administered without the addition of further pharmaceutical excipients. For example, one could process a composition of the present invention into a bacteria powder and fill said powder into hard capsules. A bacteria powder could be obtained e.g. by lyophilizing or spray-drying of a composition of the present invention. Alternatively compositions of the present invention or processing products thereof can be admixed with pharmaceutical excipients. Preferably pharmaceutical excipients are used to produce an administration form comprising a composition of the invention. Suitable pharmaceutical excipients therefor are well known to a person skilled in the art and described in the professional literature. Pharmaceutical excipients are e.g. described in the “Handbook of Pharmaceutical Excipients” by R. C. Rowe et al. Pharmaceutical excipients as used herein include, but are not limited to the following categories carriers, stabilizers, antiadherents, binders, coatings, disintegrants, fillers, flavours, colours, lubricants, glidants, sorbents, preservatives or sweeteners. In some embodiments such pharmaceutical excipients are added to or are part of compositions of the present invention.

“Pharmaceutical excipients” can also be comprised in a composition of the present invention through the method of the present invention. Such pharmaceutical excipients include e.g. a pharmaceutical acceptable culturing medium or remains thereof.

Within the present invention enteral application includes administration forms with a time release technology and/or a protective coating. Time release technologies include systems for a sustained-release, sustained-action, extended-release, timed-release, controlled-release, modified release, or continuous-release. “Protective coating” preferentially refers herein to an form that is protected by a gastro-resistant coat. In some aspects the entire dosage form is coated, e.g. a gastro-resistant capsule. In other aspects only subunits are coated, e.g. pellets within a capsule. Further possible systems to protect a composition of the present invention from e.g. pH, temperature or oxygen are not excluded and known to a person skilled in the art.

Within the present invention a composition comprises a microorganism, preferably obtainable by a method according to the present invention, which is preferably capable of binding a toxin from a pathogenic microorganism and/or capable of binding a surface receptor of a mammalian cell for a toxin from a pathogenic microorganism. A microorganism comprised by said composition may be a plurality of microorganisms, preferably obtainable by a method according to the present invention. Alternatively, a composition comprises an analog, variant or fragment of a microorganism, preferably obtainable by a method according to the present invention, wherein said analog, fragment, variant or the obtainable microorganism, is preferably capable of neutralizing a toxin and/or reducing the pathogenic effect of a pathogenic microorganism. An analog, variant or fragment of a microorganism comprised by said composition may be an analog, variant or fragment of a plurality of microorganisms, preferably obtainable by a method according to the present invention.

Pharmaceutical compositions of the present invention are for use in the treatment, amelioration and or prevention of a disease in a subject, wherein the subject may be an animal or a human. Pharmaceutical compositions of the present invention may be administered to a subject in need thereof, wherein the subject may be an animals or a human.

Whether a composition of the present invention is a pharmaceutical is preferably defined by the dosage of the composition or of processing products thereof. Without limiting the scope of the present invention a composition is to be regarded as a pharmaceutical if the ingestion of said composition in the prescribed quantity leads to a therapeutic effect.

In one aspect the present invention is a method of treating, alleviating, or preventing a gastrointestinal disease in a subject, comprising administering a therapeutically effective amount of a composition, microorganism(s), analog, variant, fragment, lysate or fraction of the present invention.

Another aspect the present invention is the use of a composition, microorganism(s), analog, variant, fragment, lysate or fraction of the present invention for treating, alleviating, or preventing a gastrointestinal disease.

A further aspect of the present invention is a kit for performing a method of the present invention, wherein said kit comprises a toxin from a pathogenic microorganism and/or a binding moiety being capable of binding a structure (A) on the surface of a naturally-occurring microorganism being capable of binding a toxin from a pathogenic microorganism.

An additional aspect of the present invention is a kit for performing a method of the present invention, wherein said kit comprises a comprising a surface receptor of a mammalian cell for a toxin from a pathogenic microorganism and/or a binding moiety being capable of binding a structure (B) on the surface of a naturally-occurring microorganism being capable of binding to a surface receptor of a mammalian cell for a toxin from a pathogenic microorganism.

It must be noted that as used herein and in the appended claims, the singular forms “a”, “an”, and “the”, include plural referents unless the context clearly indicates otherwise. Thus, for example, reference to “a reagent” or “a microorganism” includes one or more of such different reagents or microorganisms, respectively, and reference to “the method” includes reference to equivalent steps and methods known to those of ordinary skill in the art that could be modified or substituted for the methods described herein.

The embodiments described in the context of the methods of the present invention are applicable in the context of the uses of the present invention, mutatis mutandis.

The embodiments described in the context of the methods of the present invention are applicable in the context of the kits of the present invention, mutatis mutandis. Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step

### EXAMPLES

The following examples are offered to illustrate, but not to limit the invention.

### Example 1: Heat Labile Toxin

This example illustrates the identification of a microorganism(s) naturally expressing a binding moiety for the E. coli heat labile toxin (LT).

Travelers' Diarrhea (TD) is the most common infectious disease to affect travelers from industrialized countries to developing countries. Enterotoxigenic Escherichia coli (ETEC) is the single most common cause of TD in adult travelers, being responsible for 20 to 40% of all TD cases worldwide. ETEC can be estimated to cause disease in up to 10 million travelers per year. Transmission of ETEC is usually from fecal contaminated food and water. The infection occurs 10 hours to 3 days after exposure typically causing profuse watery diarrhea sometimes with low grade fever, abdominal cramping and/or vomiting. Furthermore, there is growing evidence that acute illness experienced by these visitors to developing countries can lead to more long-term health conditions, ranging from functional gastrointestinal disorder, like irritable bowel syndrome, to reactive arthritis in approximately 10 percent of individuals recovering from an episode of travelers' diarrhea.

Besides being a main cause of travelers' disease, ETEC is estimated to cause 280-400 million diarrheal episodes per year in children under 5 years of age in developing countries, resulting in 300,000 to 500,000 deaths. Due to difficulty in culturing the bacterium and the similarity of symptoms to other diarrheal diseases, these numbers are believed to be significantly underestimated. ETEC is the second leading cause of death in children less than 5 years of age in developing countries.

Responsible for the diarrhea are two enterotoxins produced by ETEC, a heat-stable (ST) and/or a heat-labile (LT) enterotoxin. Roughly one third of all ETEC strains isolated globally have been reported to be ST-only strains, one third ST/LT and one third LT-only strains. The natural cell surface receptor for LT is ganglioside GM1 (Gal-b1,3-GalNAcb1,4-(NeuAc-α2,3)-Gal-b1,4-Glc-b1,1-ceramide). The oligosaccharide part of GM1 (GM1-OS) is responsible for binding to LT.

A natural microorganism naturally expressing a binding moiety for LT may function as delivery vehicle for the surface-displayed binding moieties. It will be delivered directly to the gastrointestinal tract where it would bind the toxin(s), thereby abating, curing, treating or preventing the development of the ETEC associated diseases. Such a natural microorganism naturally expressing a binding moiety for LT would overcome most of the drawbacks presented by other available therapeutics. Unfortunately, to date no reports did describe non-pathogenic microorganisms naturally expressing a binding moiety binding a pathogenic toxin.

### Material and Methods

### Coupling the Heat Labile Toxin to Magnet Beads

Preparation of Beads

In the present example, tosylactivated DYNABEADS® M-450 (Life Technologies, UK) were used to directly link the heat labile toxin. Alternatively, other strategy (including but not limited to those described in FIG. 1) may be used to attach the toxin to magnet beads or any other carrier that can be isolated from a mixture of microorganisms. Alternatively, other binding moiety (antibody, lectins etc. . . . ) known to recognize a structure that binds the toxin may be linked to magnet beads or any other carrier.

The surface tosyl groups of beads allowed the direct covalent binding of protein-ligands via primary amino- or sulphydryl groups without any modification of the protein.

25 μl tosylactivated DYNABEADS® M-450 were placed in 1.5 ml Safe-Lock Eppendorf tubes (Eppendorf, Hamburg, Germany), washed twice with 200 μl buffer B (2.62 g NaH₂PO₄, 14.42 g Na₂HPO₄, pH 7.4) using the Dynal Magnetic Particle Concentrator-S (MPC™-S, Dynal Biotech, Oslo, Norway) and suspended in 25 μl buffer B.

Coupling of Ligands (Toxins) to Tosylactivated Beads (DYNABEADS® M-280)

Heat labile toxin was dissolved in water according to the manufacturer's recommendations (List Biological Laboratories, California, USA). In order to eliminate amino groups (Tris-) present in the initial buffer, heat labile toxin solution was dialyzed against PBS.

15 μl of dialyzed toxin solution containing 15 μg toxin were added to 25 μl DYNABEADS® (750 μg) and 10 μl of Buffer C (3M (NH₄)₂SO₄, 2.62 g NaH₂PO₄, 14.42 g Na₂HPO₄, pH 7.0) and incubated under gently agitation at 37° C. for 2 hours. Subsequently 5 μl of buffer C completed with 1% (w/v) Human Serum Albumin (HSA) were added to the suspension and incubated on a roller at 37° C. for 12-18 hours or at room temperature for 24 hours or at 4° C. for 48 hours. Non-specific and free binding sites were inactivated through addition of 500 μl Buffer D (0.88 g NaCl, 2.62 g NaH₂PO₄, 14.42 g Na₂HPO₄, 0.5% HSA (w/v), pH 7.4) and incubation at 37° C. for an additional 1 hour or at room temperature for 5 hours. In order to allow storage at 4° C., the coated beads were finally washed three times with 500 μl Buffer E (0.88 g NaCl, 2.62 g NaH₂PO₄, 14.42 g Na₂HPO₄, 0.1% HSA (w/v), pH 7.4) and re-suspended in 60 μl Buffer E and stored at 4° C. Coated beads were used within two weeks of preparation.

Labeling of Heat Labile Toxin
Biotinylation: Biotinylation was performed according to the manufacturer instructions using EZ-LINK™ micro Sulfo-NHS-LC-Biotinylation kit (Thermo Fisher Scientific Inc., Rockford, USA). Biotin excess was removed through dialyze using the Xpress Micro Dialyzer Kit (Scienova, Jena, Germany). Heat labile toxin concentration was determined through Bradford analysis.
Horseradish peroxidase (HRP) labelling: Horseradish peroxidase (HRP) labelling was preformed according to the manufacturer instructions using EZ-LINK™ Plus Activated Peroxidase Kit (Thermo Fisher Scientific Inc., Rockford, USA).

GM₁ Enzyme-Linked Immunosorbent Assay (ELISA).

50 μl purified Monosialoganglioside (GM1, 0.5 μg/ml in phosphate-buffered saline [PBS], pH 7.2; Sigma, Hannover, Germany) were added per well of a Maxisorp ELISA plate (Nunc, Roskilde, Denmark) and adsorbed overnight at 4° C. Alternatively, 50 μl purified Monosialoganglioside were added per well of a Polysorp ELISA plate (Nunc, Roskilde. Denmark) and adsorbed overnight at 37° C. Coated wells were washed 3 times with 200 μl PBS+0.02% Tween 20 (Identical washing steps were performed after each incubation step.) followed by 30 minutes incubation with 200 μl of PBS+2% HSA for blocking. After one wash, 50 μl of biotinylated toxin at indicated concentration were added for 2 hour at room temperature or 37° C. The plate was then washed three times with PBS+0.02% Tween 20 and incubated with 50 μl HRP-Streptavidin (1:15,000) (Streptavidin horseradish peroxidase; Sigma) for 1 hour at 4° C. or room temperature.

Alternatively, ELISA plates were incubated with 50 μl of HRP-labelled heat labile toxin or HRP-labelled heat labile toxin subunit B for 2 hours at room temperature or 37° C. After three final washes, 100 μl of TMB One Component HRP Microwell Substrate (3,3′,5,5′-tetramethylbenzidine, pH 4.5, Tebu-bio, France) was added as substrate, reaction was stopped by addition of 50 μl 2.5 N H₂SO₄ and extinction was measured at E450/630 nm (ELISA Reader, Dynex Technologies Inc., Chantilly, USA).

Optionally, to confirm the carbohydrate specificity of the binding, coated GM1 may be submitted to enzymatic or chemical treatment affecting the structure of the carbohydrate. As an example, mild (carbohydrate-specific) periodate oxidation (PI) was used prior to incubation with biotinylated heat labile toxin. Periodate oxidation preferably cleaves terminal sugar rings with vicinal hydroxyl groups. Therefore, a decreased intensity of binding following mild periodate oxidation demonstrate the binding to be carbohydrate dependent. Coated wells were incubated with 50 μl of 50 mM sodium acetate buffer (pH 4.5) for 5 min. Next, 50 μl per well of 10 mM sodium periodate (Sigma, Hannover, Germany) in sodium acetate buffer was added and incubated for 1 h in the dark. After 3 washes, 50 μl sodium acetate buffers was added for 5 min followed by a 30 min incubation with 50 μl of 50 mM borohydride (Sigma) in PBS to block the sodium periodate. The plates were washed five times and the ELISA was carried out as described above.

Procedure for Testing the Capacity of Labeled Toxin to Bind its Natural Receptor on the Surface of Human Cells.

Cytotoxic Cellular Assay

Human colorectal adenocarcinoma cell line HT-29 were seeded in 96 well cell culture plates at concentrations between 2×10⁴ and 1×10⁵ cells/well and cultivated for at least 2 days in standard McCoy Medium added with 10% FBS and 2 mM butyrate. The presence of butyrate induced the differentiation of HT29 cells (48), thereby increasing the expression of toxin-receptor on the surface of the cells. 20 μl of a solution of heat-labile toxin at indicated concentration labeled or not were added per well and the cellular morphological phenotype monitored after 4, 16 and/or 30 hours.

Preparation of Samples

1 g of a mixture of microorganisms (e.g. fresh feces sample from healthy adults) were suspended in a tube containing 9 ml of anaerobic PBS_{red} (8.5 g/l NaCl, 0.6 g/l Na₂HPO₄, 0.3 g/l KH₂PO₄, 0.25 g/l Cystein.HCl, 0.1 g/l Peptone, pH 7.0) and stored at 4° C. in an anaerobic box (Anaerogen, Oxoid, Wesel, Germany). The samples were processed within 4 hours in an anaerobic chamber as followed: sterile 3 mm diameter glass beads were added and the samples were homogenized by vortexing. The homogenized fecal suspensions were centrifuged (300×g for 1 min) to sediment debris. The resulting supernatant was transferred in a new tube and centrifuged again. The supernatant was either diluted 1:10 (v/v) with anaerobic PBS_{red}+/−0.1% BSA (or HSA) and used directly or frozen at −20° C. for later use. Alternatively, the fresh samples may be stored and processed under non-anaerobic conditions, allowing the isolation of aerobic or facultative anaerobic microorganisms.

Optionally, the fecal sample may be depleted or enriched in any specific genus or species e.g. by mean of affinity depletion/enrichment, antibiotic treatment or any alternative method. For example, in order to increase the proportion of Bifidobacterium and Lactobacillus in fecal sample, the sample was further depleted in Bacteroides by mean of centrifugations. Bacteroides are the most abundant genus of the human colonic microbiota, surpassing Lactobacillus and Bifidobacterium by a factor 10,000. In order to increase the proportion of Bifidobacterium and Lactobacillus in fecal sample, the feces suspensions were centrifuged further for 3 min at 2500 rpm. Due to their small size, Bacteroides were mostly restricted to the supernatant that was discarded. The pellet was re-suspended in PBS_{red} and the procedure repeated three times. The final bacteria pellet was suspended in 1 ml anaerobic PBS_{red}+0.1% BSA or HSA.

Isolation of Microorganism Naturally Binding Heat Labile Toxin

Isolation of Bacteria Using Toxin-Coated Beads

20 μl of bacterial suspension were added to 175 μl PBS_{red}+0.1% BSA or HSA and 5 μl “heat labile toxin”-coated beads. The mix was incubated at room temperature (RT) or 37° C. under appropriate atmosphere and gently agitation for 1 hour. Subsequently, the beads were washed twice with 200 μl PBS_{red}+0.01% BSA or HSA. To reduce non-specific binding of viable bacteria, the beads may be pre-incubated for 1 hour with pasteurized bacteria (e.g. Bifidobacterium spp.) at RT or 37° C. Pasteurized bacteria may also be added during the incubation of viable bacteria with the heat labile toxin-coated beads.

After incubation the beads were washed and re-suspended in 1 ml PBS_{red}. 100 μl aliquots were spread-plated on unspecific and specific agar plates like e.g. WC agar (Oxoid, Wesel, Germany), MRS agar (Merck, Darmstadt, Germany) and Bifidus Selective agar (BSM, Fluka, St. Gallen, Switzerland). All plates were incubated under appropriate conditions. Well isolated Colonies were picked randomly from agar plates. Optionally, the colonies may be streaked several times on nonselective or selective media. Alternatively, isolated colonies may be pooled together and submitted again (several times) to the isolation process.

Growth, Inactivation and Maintenance of Isolates

Well separated colonies were randomly picked from agar plates, inoculated into corresponding broth mediums and grown under appropriate conditions. The resulting cultures were partly used for production of cryo-stocks and partly used for future screening analysis. Cryo-stocks were produced by addition of 1:1 (v/v) of a 30% (v/v) glycerol solution in appropriate medium and stored at −80° C. For screening analysis, bacteria cultures were centrifuged, washed with PBS and re-suspended in PBS. Part of the re-suspended bacteria was inactivated through pasteurization in water bath at 75° C. for 15 min or through or UV-treatment (e.g. at 254 nm for 400 seconds). As a control for successful inactivation bacteria were plated on a suitable agar medium. Washed and washed and inactivated bacteria were stored at 4° C.

Identification of Isolates

Preliminary identification of isolated bacteria was based on microbiological analysis (e.g. Gram staining, microscopic analysis etc. . . . ) and biochemical analysis (e.g. with rapid ID 32A biochemical test kits (BioMerieux, Marcy I'Etoile, France)).

Alternatively, the characterization was performed by Bruker Biotyper (version 2.0) matrix-assisted laser desorption ionization-time of flight (MALDI-TOF) mass spectrometry. Briefly, colonies were directly picked and applied as a thin film onto a polish steel plate and allowed to dry at room temperature. Subsequently, 1 μl of MALDI matrix (Bruker Daltonics) in 50% acetonitrile and 2.5% trifluoroacetic acid was applied and allowed to dry again.

For the extraction method, 1 to 2 colonies (or a few colonies in the case of a small colony size) were suspended in 300 μl of molecular-grade water (Sigma-Aldrich, St. Louis, Mo.) and vortexed. Next, 900 μl of 100% ethanol (Sigma-Aldrich) was added, vortexed, and centrifuged (13,400×g) for 2 min. The supernatant was decanted, and the pellet was dried at room temperature. 10 μl of 70% formic acid (Fluka [Sigma-Aldrich], St. Louis, Mo.) and 10 μl of acetonitrile (Fluka) were added and thoroughly mixed by pipetting, followed by centrifugation (13,400×g) for 2 min. One microliter of supernatant was spotted onto the 384-spot plate and allowed to dry at room temperature before the addition of 1 μl of matrix. For each plate, a bacterial test standard (Bruker Daltonics) was included to calibrate the instrument and validate the run.

MALDI-TOF MS was performed with the MicroFlex LT mass spectrometer (Bruker Daltonics) according to the manufacturer's suggested recommendations. Identification score criteria used were those recommended by the manufacturer: a score of 2.000 indicated species-level identification

Screening of Bacterial Strains Binding Heat Labile Toxin

Procedure for Testing the Capacity of Isolates to Bind the Heat Labile Toxin

For standardization of assays, the absorbance of previously washed cultures (inactivated or not inactivated) was measured at 600 nm (A600). The bacterial solutions were than adjusted to appropriate absorbance (i.e. 0.5 or 1) with PBS and 50 μl of bacterial solution were coated per well of a 96 well Polysorp microtiter plate (Nunc, Roskilde, Denmark) and incubated overnight at 37° C. Alternatively, the protein concentration of the washed cultures (inactivated or not inactivated) was measured. Protein concentration was determined by the Bradford method using the BioRad assay reagent (Bio-Rad, Munich, Germany). A bovine serum albumin (BSA) standard curve was used to calculate the protein concentration. Bacterial solutions were adjusted to appropriate protein concentration (i.e. 5 μg protein per ml) with PBS and 50 μl of bacterial solution were coated per well of a 96 well polysorp microtiter plate (Nunc, Roskilde, Denmark) and incubated overnight at 37° C.

Alternatively, the bacterial concentration (cells/ml) was determined microscopically and 50 μl of bacterial solution at identical cell concentration (i.e. 1×10⁸ cells/ml) were coated per well of a 96 well polysorp microtiter plate (Nunc, Roskilde, Denmark) and incubated overnight at 37° C.

Alternatively, the bacterial solutions were coated without prior standardization. The concentrations of the bacterial solutions were determined afterward as mentioned above and the results (ELISA signals) standardized to the appropriate A600, Protein concentration or cell number.

Plates were washed 3 times with PBS+0.02% Tween 20 and blocked with 200 μl PBS+2% human serum albumin (HSA) or bovine serum albumin (BSA). The plates then were incubated with 50 μl of 100 ng/ml biotinylated heat labile toxin or biotinylated thyroglobulin as negative control for 2 hours at room temperature or 37° C. The plate was then washed three times with PBS+0.02% Tween 20 and incubated with 50 μl HRP-Streptavidin (1:15,000 in PBS) (Streptavidin horseradish peroxidase; Sigma).

Alternatively, the plates were incubated with 50 μl of 100 ng/ml HRP-labeled heat labile toxin or HRP alone as negative control for 2 hours at room temperature or 37° C.

After three final washes, the assay was developed with TMB as substrate, reaction was stopped by addition of 2.5 N H₂SO₄ and extinction was measured at 450/630 nm (as detailed above).

To confirm the involvement of carbohydrate structure(s) in the binding of the heat labile toxin to isolated microorganisms, coated bacteria were submitted to mild periodate oxidation (PI) as described above prior to incubation with heat labile toxin.

Results

Binding Activity of Labeled Heat Labile Toxin

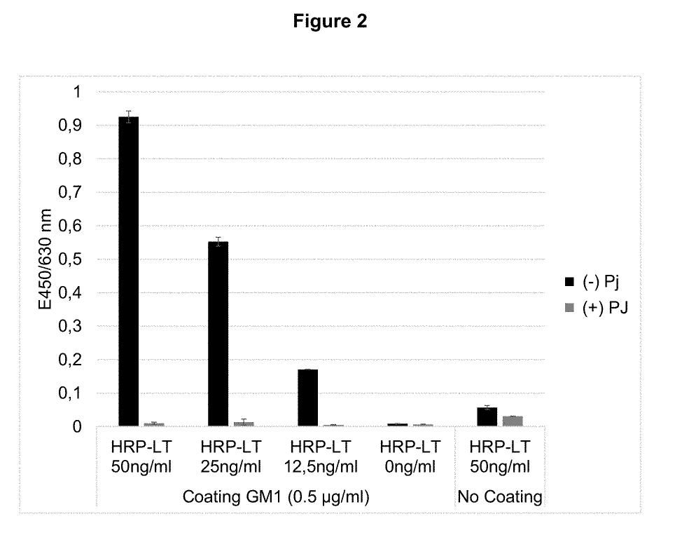

The ability of labeled LT and labeled LT subunit B to bind their natural receptor monosialoganglioside (GM1) by means of an ELISA was analyzed. As presented in FIG. 2, HRP-labelled LT bound its natural carbohydrate receptor GM1 in a concentration dependent manner. Furthermore, the mild periodate oxidation of GM1 prior to incubation with HRP-labeled heat labile toxin resulted in a reduction of the signal previously observed, confirming the carbohydrate specificity of the interaction. Similar results were obtained for biotinylated-LT, confirming that the labeling (biotinylation or HRP-labeling) did not prohibit the natural binding activity of the heat labile toxin.

The ability of unlabeled and labeled (biotinylated or HRP-labeled) heat labile toxin to bind its natural receptor on the surface of HT-29 cells was analyzed by mean of cytotoxic cellular assay.

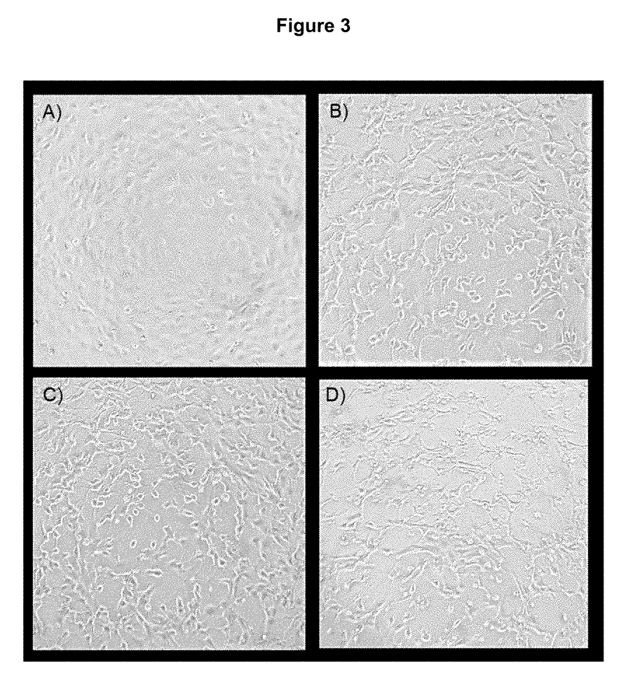

HT-29 cells cultured for at least 2 days in medium containing 2 mM butyrate were strongly sensitive to the heat labile toxin. Unlabeled and labeled heat labile toxin presented identical concentration dependent cytotoxicity effect on HT-29 cells. Thus, more than 50% of HT-29 cells presented morphological modifications (irregular outline and numerous microspikes) after 16 hours of incubation with 0.5 ng/ml heat labile toxine (FIG. 3), demonstrating that labeled heat labile toxin conserved its ability to bind its natural receptor on the surface of HT29 cells and its ability to induce cytotoxicity.

Binding of Heat Labile Toxin and Periodate Sensitivity

The capacity of isolated commensal bacteria to bind heat labile toxin by means of an enzyme-linked immunosorbent assay (ELISA) using labeled-heat labile toxin was studied. Strong natural binding capacity for the heat labile toxin was a rare event.

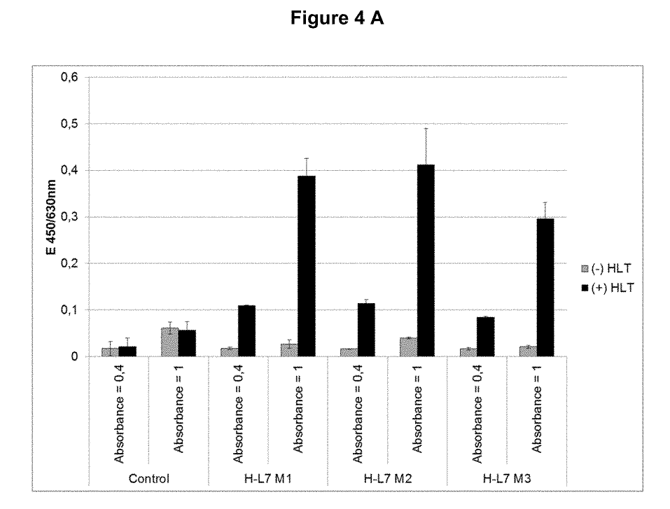

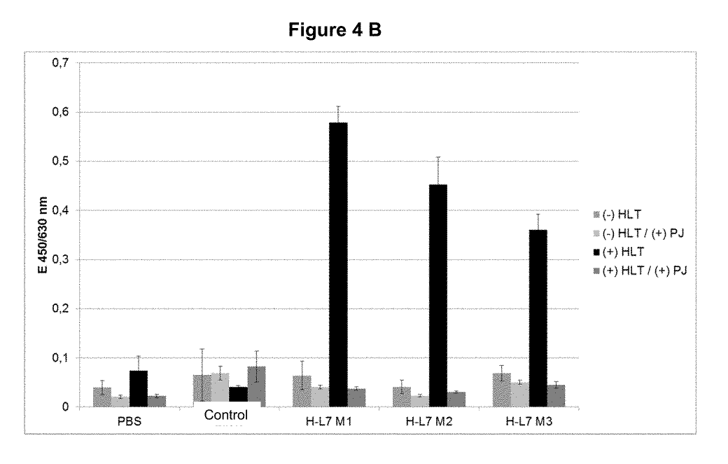

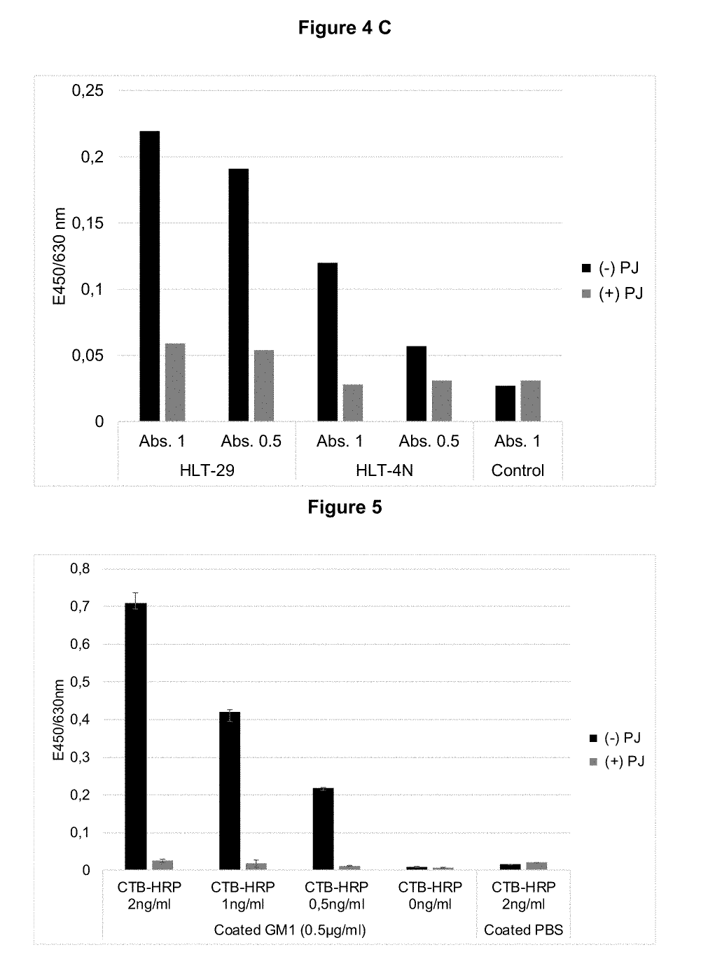

Five strains presented a strong and dose dependent natural binding capacity for heat labile toxin (FIG. 4A to 4D). The toxin binding property was related to the cells itself as it was conserved despite successive centrifugations and wash steps of the cells. Furthermore, for all isolates the binding was reduced by prior mild periodate oxidation of the coated bacteria, confirming the involvement of a carbohydrate structure in the binding of heat labile toxin (FIG. 4).

The strains HLT-L7M1, HLT-L7M2 and HLT-L7M3 present more than 99% biochemical similarity to Lactobacillus spp., the strain HLT-4N was characterized as belonging to the species Lactobacillus paracasei and the strain HLT-29 was identified as an E. coli strain.

Conclusion

The present study is the first one to report the isolation out of the human gut microflora of microorganisms naturally expressing a specific binding moiety for the E. coli heat labile toxin. Four Lactobacillus strains and one E. coli strain presenting a strong and dose dependent binding of the heat labile toxin were isolated.

The binding of LT was further sensitive to mild periodate oxidation, suggesting that the binding moiety expressed on the surface of the bacteria contains a carbohydrate structure directly involved in the binding.

These surprising results unequivocally demonstrate that the invention allows isolating natural inhabitants of the human flora that are naturally expressing a binding moiety for the E. coli heat labile toxin. Such microorganisms may be developed as drug or food supplement to be administered orally to human or animal in viable or killed form to abate, cure, treat or prevent diseases related with E. coli heat labile toxin.

### Example 2: Cholera Toxin

This example illustrates the identification of microorganisms naturally expressing a binding moiety for the Cholera Toxin (CT).

Cholera is an acute, diarrheal illness caused by infection of the intestine with the bacterium Vibrio cholerae. An estimated 3 to 5 million cases and over 100,000 deaths occur each year around the world (46). Heat labile toxin (LT) from ETEC and Cholera toxin are highly identical and both were reported to bind the same carbohydrate receptor GM1 described above. However, slightly different specificities were reported (47, 48).

A natural microorganism naturally expressing a binding moiety for CT may function as delivery vehicle for the surface-displayed binding moieties. It will be delivered directly to the gastrointestinal tract where it would bind the toxin(s), thereby abating, curing, treating or preventing the development of the Vibrio cholerae associated diseases. Such a natural microorganism naturally expressing a binding moiety for CT would overcome most of the drawbacks presented by other available therapeutics. Unfortunately, to date no reports did describe non-pathogenic microorganisms naturally expressing a binding moiety binding a pathogenic toxin.

Material and Methods

Coupling the Cholera Toxin to Magnet Beads

Preparation of Beads

In the present example, DYNABEADS® M-270 Amine (Life technologies, UK) were used to directly link the Cholera toxin (List Biological Laboratories, Inc). The Surface-reactive primary amino-groups allow immobilization of ligands such as carbohydrates, glycoproteins and glycolipids through reductive amination of aldehyde or ketone groups. Alternatively, ligands can be immobilized through amide-bond formation with carbodiimide-activated carboxylic acid groups. Bi-functional cross-linkers may be used to introduce other functional groups.

Alternatively, as mentioned in example 1 other strategy may be used to attach the toxin or another binding moiety (antibody, lectins etc.) known to recognize a structure that binds the toxin to magnet beads or any other carrier that can be isolated from a mixture of microorganisms.

25 μl of DYNABEADS® M-270 Amine were placed in 1.5 ml Safe-Lock Eppendorf tubes (Eppendorf, Hamburg, Germany), washed twice with 100 μl buffer F (0.26 g NaH₂PO₄+H₂O, 1.44 g Na₂HPO₄+2H₂O, 8.78 g NaCl, pH 7.4) using the Dynal Magnetic Particle Concentrator®-S (MPC®-S, Dynal Biotech, Oslo, Norway) and suspended in 25 μl buffer F. Before coupling of ligands to Beads the washed DYNABEADS® were activated with NHS (N-hydroxy-succinimidyl)-ester cross-Linker. Hereby the beads were re-suspended in 0.1 M sodium phosphate buffer with 0.15 M NaCl, pH 7.4. Dissolved NHS-ester (15 mg/ml) was added to the bead-solution. Beads were incubated for 30 min at room temperature with slow tilt and rotation, and finally washed twice with buffer F and re-suspended in 25 μl buffer F.

Preparation of Used Ligands (Toxins) for Coating the DYNABEADS®

Cholera toxin was dissolved in water according to the manufacturer's recommendations (List Biological Laboratories, California, USA). In order to eliminate amino groups (Tris-) present in the initial buffer, Cholera toxin solution was dialyzed against PBS.

Coupling of Ligands (Toxins) to DYNABEADS® M-270 Amin (Life Technologies, UK)

For coupling of carboxyl-groups of ligand (Toxin) to Amine-Group of beads the EDC (1-(3-Dimethylaminopropyl)-3-ethylcarbodiimid) or a mix of EDC/NHS was used.

5 μl of beads were washed once with 100 μl of 0.1 M MES (2-[N-morpholino] ethane sulfonic acid) and 0.5 M NaCl at pH 6 and re-suspended in 100 μl MES. 5 μl of dialyzed toxin solution containing 2 μg toxin were added and gently mixed. 10 μl of EDC (10 mg/ml) or EDC/NHS (10 mg/ml EDC and 15 mg/ml NHS) were added to the beads-toxin suspension, mixed gently and incubated for 2 hours at room temperature with slow tilt rotation. 10 mM of hydroxylamine (NH₂OH×HCl) were added to quench the reaction, and incubated for 15 min at room temperature with slow tilt rotation. Coated beads were washed twice with buffer F and re-suspended in 5 μl buffer F and stored at 4° C. Coated beads were used within two weeks of preparation.

Labeling of Cholera Toxin
Horseradish peroxidase (HRP) labelled Cholera Toxin Subunit B was purchased from (Thermo Fisher Scientific Inc., Rockford, USA).
Alternatively, the whole toxin was labelled with HRP: Horseradish peroxidase (HRP) labelling was preformed according to the manufacturer instructions using EZ-Link™ Plus Activated Peroxidase Kit (Thermo Fisher Scientific Inc., Rockford, USA).
His-tagged Cholera toxin subunit B: In the present example, a 6 Histidine amino acids sequence was tagged to the C-terminal end of the Cholera toxin subunit B. The cDNA of Cholera toxin subunit B was amplified by PCR, purified and cloned into an E. coli expression vector containing a 6× Histidine tag. After usual transformation of competent E. coli cells, expressing cells were selected, inoculated in LB Medium and grown at 37° C. at 200 rpm until the culture reached an OD₆₀₀ approx. 0.4-0.8. Expression of his-tagged Cholera toxin subunit B was induced by addition of 40-400 μM Isopropyl 1-D-1-thiogalactopyranoside (IPTG) and incubated further for 3 hours. The cells were then harvested by centrifugation at 4° C. and re-suspended in the lysis buffer and incubated on ice for 30 min.

Cells were then lysed by sonication and the lysate was cleared by centrifugation. The supernatant was purified using immobilized metal-affinity chromatography (IMAC) column matrix immobilized with Ni²⁺. The running buffer was used containing 50 mM Tris, 100 mM NaCl and 20 mM Imidazole, pH 7.5. The elution of his tagged toxin was carried out in 20-500 mM Imidazole and the collected elution fractions were analyzed on a 12% SDS-PAGE. The collected purified His-Tagged toxin subunit B was stored at −80° C.

GM₁ enzyme-linked immunosorbent assay (ELISA) was performed as described in example 1.

Procedure for Testing the Capacity of Labeled Toxin to Bind its Natural Receptor on the Surface of Human Cells.

Cytotoxic Cellular Assay

Cytotoxicity Cellular Assay was Performed as Described in Example 1.

Isolation of Microorganism Naturally Binding Cholera Toxin

Isolation of Bacteria using toxin-coated beads was performed as described in example 1. Alternatively, the bacterial solution was first incubated with a tagged toxin or tagged subunit of the toxin and subsequently with Magnetbeads specifically directed against the Tag (DYNABEADS® His-Tag. Life technologies, UK). In our example, the bacterial solution was first incubated with the C-terminal His-Tagged subunit B of the toxin. 200 μl of bacterial suspension were added to 800 μl PBS_{red}+0.1% BSA or HSA and 2 μg “His-tagged Cholera Toxin Subunit B”. The mix was incubated at room temperature (RT) or 37° C. under appropriate atmosphere and gently agitation for 1 hour. Subsequently, 5 μl of DYNABEADS® His-Tag was added to the mix. Other incubation conditions were performed as described in example 1. After incubation the beads were washed and re-suspended in 1 ml PBS_{red}. 100 μl aliquots were spread-plated on unspecific and specific agar plates as described in example 1.

Growth, Inactivation, Maintenance and Characterization of Isolates were Performed as Described in Example 1.

Screening of Bacterial Strains Binding the Cholera Toxin

Procedure for Testing the Capacity of isolates to bind the Cholera toxin Standardization of assays and coating of the polysorp microtiter plate (Nunc, Roskilde, Denmark) were performed as described in Example 1.

Plates were washed 3 times with PBS+0.02% Tween 20. After blocking of plates with 200 μl PBS+2% human serum albumin (HSA) or bovine serum albumin (BSA), ELISA plates were incubated with 50 μl of 100 ng/ml HRP-labelled Cholera Toxin Subunit B for 2 hour at room temperature or 37° C. The plate was then washed three times with PBS+0.02% Tween 20 the assay was developed with TMB as substrate, reaction was stopped by addition of 2.5 N H₂SO₄ and extinction was measured at 450/630 nm (as detailed above).

To confirm the involvement of carbohydrate structure(s) in the binding of the Cholera Toxin to isolated microorganisms, coated bacteria were submitted to mild periodate oxidation (PI) as described in Example 1 prior to incubation with heat labile toxin.

Testing the Toxin Neutralization Potential of Cholera Toxin Binding Strains

Inhibition of CT binding to differentiated human colon cell line HT-29 (Competition ELISA) Here, the ability of isolated CT-binding strains to inhibit the binding of the CT to human intestinal cells was analyzed. Human colorectal adenocarcinoma cell line HT-29 were cultivated for 4 days in standard McCoy Medium added with 10% FBS and 2 mM butyrate. HT-29 were detached from the culture flask through the action of accutase. The cells were allowed to recover in culture Medium containing 10% FBS and 2 mM with butyrate for 2 hours before being washed and fixed with 2.5% paraformaldehyde for 2 hours at room temperature. Cells were washed three time end re-suspended in PBS. 50 μl of fixed HT-29 cells (5×10⁴ to 5×10⁵ cells/ml) were added per well of a polysorp ELISA plate (Nunc, Roskilde, Denmark) and dried at 37° C. overnight.

Plates were washed 3 times with PBS+0.02% Tween 20. After blocking of plates with 200 μl PBS+2% HSA, ELISA plates were incubated with 50 μl of 10 ng/ml HRP-labelled Cholera Toxin subunit B or with 10 ng/ml HRP-labelled Cholera Toxin subunit B mixed with CT-binding bacteria (1×10⁷ to 5×10⁸ cells/ml) for 2 hour at room temperature or 37° C. A bacterial strain that do not bind CT was used as negative control. The plate was then washed three times with PBS+0.02% Tween 20 the assay was developed with TMB as substrate, reaction was stopped by addition of 2.5 N H₂SO₄ and extinction was measured at 450/630 nm (as detailed above).

Results

Binding Activity of HRP Labelled Cholera Toxin

The ability of HRP-cholera toxin and HRP-cholera toxin subunit B to bind their natural receptor monosialoganglioside (GM1) by means of an ELISA was analyzed. As presented in FIG. 5, the HRP-cholera toxin subunit B bound its natural carbohydrate receptor GM1 in a concentration dependent manner. Furthermore, the mild periodate oxidation of GM1 prior to incubation with HRP-Cholera toxin Subunit B resulted in a strong reduction of the signal, confirming the carbohydrate specificity of the interaction. Similar results were obtained with the HRP-labeled Cholera toxin.

The ability of unlabeled and HRP-labelled Cholera Toxin to bind its natural receptor on the surface of HT-29 cells was analyzed by mean of cytotoxic cellular assay.

HT-29 cells cultured for at least 2 days in medium containing 2 mM butyrate were strongly sensitive to the heat labile toxin (See example 1). Unlabeled and HRP-labeled Cholera toxin presented identical concentration dependent cytotoxicity effect on HT-29 cells, demonstrating that HRP-labeled cholera toxin conserved its ability to bind its natural receptor on the surface of HT29 cells and to induce cytotoxicity.

Binding of Cholera Toxin and Periodate Sensitivity

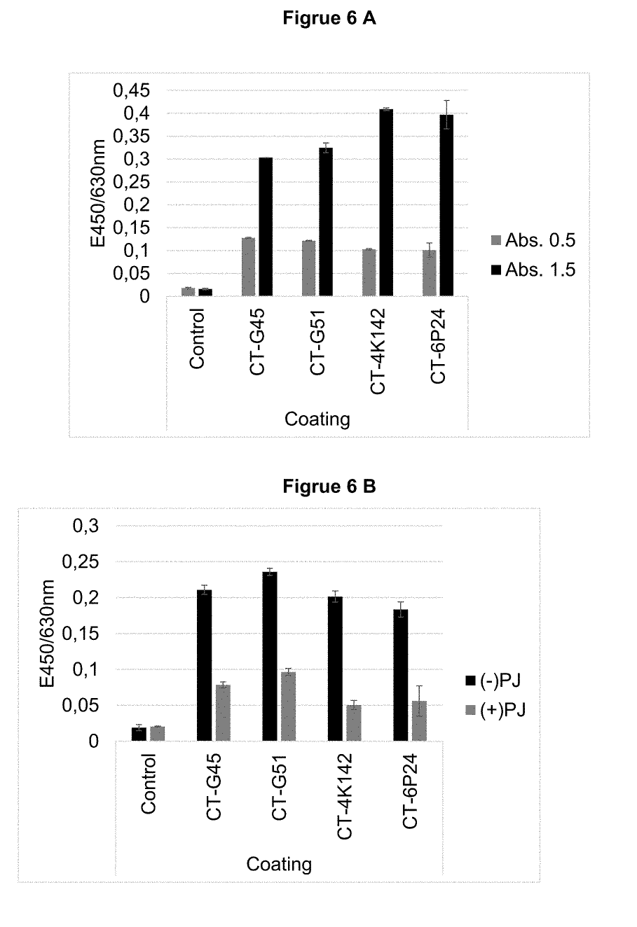

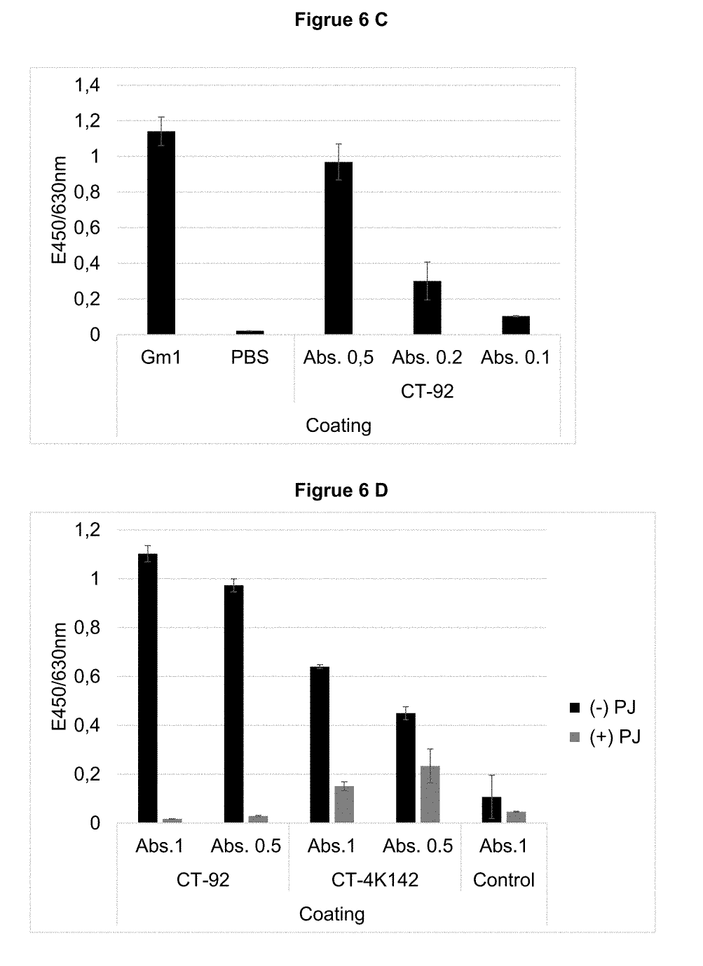

The capacity of isolated commensal bacteria to bind Cholera toxin by means of an enzyme-linked immunosorbent assay (ELISA) using HRP-cholera toxin subunit B was studied. Five strains presented a strong and dose dependent natural binding capacity for Cholera toxin (FIG. 6 a to D). Furthermore, for all isolates the binding was reduced by prior mild periodate oxidation of the coated bacteria, confirming the involvement of a carbohydrate structure in the binding of heat labile toxin.

The strains CT-G45 and CT-G51 were characterized as belonging to the species Lactobacillus reuteri, strains CT-4K142 and CT-6P24 were characterized as belonging to the species Lactobacillus paracasei and the strain CT-92 was identified as an Enterococcus faecalis strain (see table 1).

TABLE 1
Preliminary taxonomic characterization of isolated strains
The table presents the best match, the second-best match and
if any, the first alternative match obtained for the taxonomic
characterization run with the Bruker Biotyper.
Table 1: Results Biotyper.

| | | | Organism | | Organism | |
|---|---|---|---|---|---|---|
| | Organism (best | Score | (second best | Score | (Alternative | Score |
| Strain | match) | Value | match) | Value | match) | Value |
| HLT- | Lactobacillus | 2.409 | Lactobacillus | 2.338 | N.A. | |
| 4N | paracasei | | paracasei | | | |
| HLT-29 | Escherichia coli | 2.377 | Escherichia coli | 2.292 | N.A. | |
| CT- | Lactobacillus | 2.282 | Lactobacillus | 2.269 | Lactobacillus | 1.641 |
| G45 | reuteri | | reuteri | | oris | |
| CT- | Lactobacillus | 2.308 | Lactobacillus | 2.249 | N.A. | |
| G51 | reuteri | | reuteri | | | |
| CT- | Lactobacillus | 2.477 | Lactobacillus | 2.468 | N.A: | |
| 4K142 | paracasei | | paracasei | | | |
| CT- | Lactobacillus | 2.435 | Lactobacillus | 2.317 | N.A. | |
| 6P24 | paracasei | | paracasei | | | |
| CT-92 | Enterococcus | 2.467 | Enterococcus | 2.409 | N.A. | |
| | faecalis | | faecalis | | | |
| STx1- | Citrobacter | 2.338 | Citrobacter | 2.313 | Citrobacter | 2136 |
| P16B7 | freundii | | freundii | | braakii | |
| Stx1- | Klebsiella | 2.314 | Klebsiella | 2.244 | Raoultella | 2.185 |
| P20F4 | oxytoca | | oxytoca | | planticola | |
| Stx1- | Klebsiella | 2.296 | Klebsiella | 2.279 | Raoultella | 2.014 |
| P21A5 | oxytoca | | oxytoca | | planticola | |
| Stx1- | Klebsiella | 2.302 | Klebsiella | 2.252 | Raoultella | 2.150 |
| P21E8 | oxytoca | | oxytoca | | planticola | |
| STx2- | Citrobacter | 2.429 | Citrobacter | 2.385 | Citrobacter | 2.290 |
| 10D1 | freundii | | freundii | | braakii | |
| Stx2- | Enterococcus | 2.462 | Enterococcus | 2.417 | N.A. | |
| 10-D7 | faecalis | | faecalis | | | |
| Stx2- | Enterococcus | 2.468 | Enterococcus | 2.456 | N.A. | |
| 15-A3 | faecalis | | faecalis | | | |

N.A. => the software did not deliver any alternative match.

Interestingly, the strain CT-6P24 and CT-92 were also able to bind the Heat-labile toxin. Furthermore, strains HLT-4N and HLT-29 (example 1) were also able to bind the cholera toxin.

Inhibition Potential

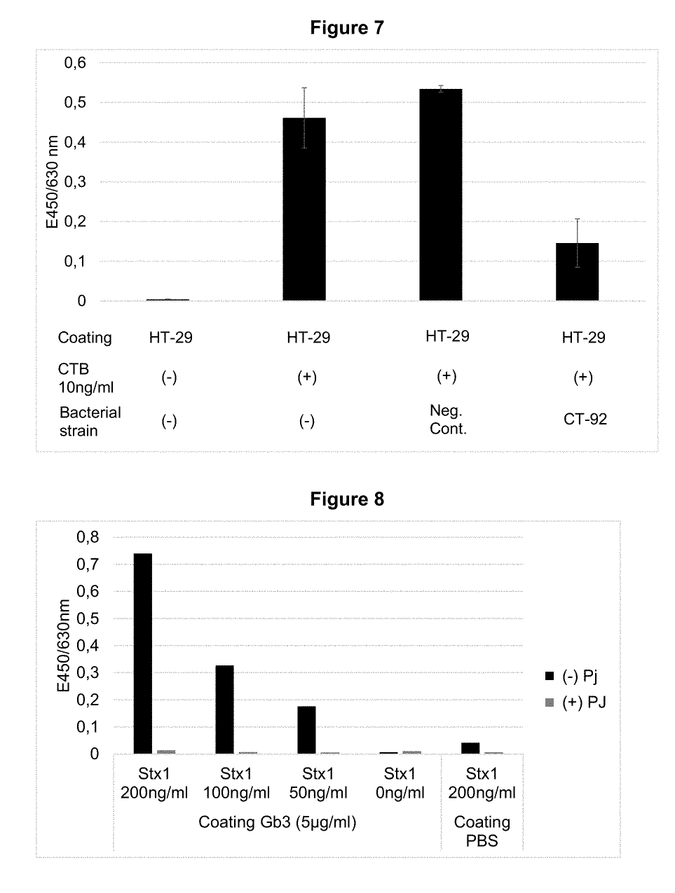

The ability of CT-binding strains to inhibit the binding of HRP-cholera toxin to human colon cell line HT-29 was analyzed by mean of a competition ELISA. As shown in FIG. 7, the addition of 1×10⁸ cells/ml of the CT-binding strain CT-92 induced an 80% reduction of the CT-HRP binding to HT29 cells, demonstrating that our CT-binding strain is able to displace CT from human CT-binding intestinal cells.

Conclusion

The present study is the first one to report the isolation out of the human gut microflora of microorganisms naturally expressing a specific binding moiety for the Cholera Toxin.

Four Lactobacillus and one Enterococcus faecalis strains presenting a strong and dose dependent binding of the cholera toxin were isolated. The binding of CT was further sensitive to mild periodate oxidation, suggesting that the binding moiety expressed on the surface of the bacteria contains a carbohydrate structure directly involved in the binding. Furthermore, cholera binding strains were able to displace CT from human CT-binding colorectal cells in an ELISA competition assay.

These surprising results unequivocally demonstrate that the invention allows isolating natural inhabitants of the human flora that are naturally expressing a binding moiety for the Cholera Toxin. Such microorganisms may be develop as drug or food supplement to be administered orally to human or animal in viable or killed form to abate, cure, treat or prevent diseases related with Cholera toxin.

### Example 3: Shiga Toxin

The most common sources for Shiga toxin are the bacteria Shigella dysenteriae and the Shigatoxigenic group of Escherichia coli (STEC). STEC accounts for an estimated 314,000 infections annually in industrialized countries, including approximately 110,000 people in the United States and 97,000 people in the European Union according to the Centers for Diseases Control and Prevention (CDC) and the (EMEA), respectively. Shigella also causes approximately 580,000 cases annually among travelers and military personnel from industrialized countries.

Symptom induced by STEC may be restricted to mild diarrhea but can evolve to hemorrhagic colitis, and potentially to life-threatening Hemolytic Uremic Syndrome (HUS). HUS is characterized by hemolytic anemia, thrombic thrombocytopenia, and renal failure. About 5% to 20% of STEC infected individuals may develop HUS (CDC). HUS presents a 5% to 10% fatality rate and survivors may have lasting kidney damage (30, 31, 32).

The most important virulence factors responsible for the evolution of the complications are the Shiga toxin Stx1 and Stx2. HUS is induced by the systemic action of Stx2 on the kidney (33). During the early stages of human infections, STEC may colonize the gut at high levels, exposing the host to sustained high concentrations of Stx and increasing the likelihood of systemic complications. As disease progresses, the numbers of STEC decrease markedly due to response of the immune system. However, this response occurs too slowly to prevent Stx-induced complications.

Both Stx1 and Stx2 toxins bind to the eukaryotic carbohydrate receptor globotriaosyl ceramide (Gb₃) (or Gb₄ as is the case for Stx2e). Stx2 that binds preferentially to Gb3 variants in kidney tissue is associated with more severe disease outcome and is 100- to 400-fold more potent than Stx1 (33).

Natural microorganisms naturally expressing binding moieties for Stx1 and Stx2 may function as delivery vehicle for the surface-displayed binding moieties. They will be delivered directly to the gastrointestinal tract where they would bind the toxins, thereby abating, curing, treating or preventing the development of the STEC associated diseases. Such natural microorganisms naturally expressing binding moieties for Stx1 and Stx2 would overcome most of the drawbacks presented by other available therapeutics. Unfortunately, to date no reports did describe non-pathogenic microorganisms naturally expressing a binding moiety binding a pathogenic toxin.

Material and Methods

Coupling the Shiga Toxins to Magnet Beads

Preparation of Beads

In the present example, the Shiga toxin 1 or the Shiga Toxin 2 (Tufts Medical Center, Phoenix Laboratory, Boston, USA) were directly linked to DYNABEADS® M-270 Amine (Life technologies, UK) as described in example 2.

Alternatively, other strategy as mentioned in example 1 may be used to attach the toxin or other binding moiety (antibody, lectins etc. . . . ) known to recognize a structure that binds the toxin to magnet beads or any other carrier that can be isolated from a mixture of microorganisms.

Labeling of Shiga toxins
Shiga Toxin 1 and 2 (Tufts Medical Center, Phoenix Laboratory, Boston, USA) were labelled with Horseradish peroxidase (HRP) according to the manufacturer instructions using EZ-LINK™ Plus Activated Peroxidase Kit (Thermo Fisher Scientific Inc., Rockford, USA).
His-Tagged Subunit B of Shiga Toxin 1 and 2. Production and purification of His-tagged Subunit B of Shiga toxin 1 and 2 were preform as described in example 2.

Gb3 Enzyme-Linked Immunosorbent Assay (EL/SA).

50 μl purified globotriaosylceramide (Gb3, 5 μg/ml in phosphate-buffered saline [PBS], pH 7.2; Sigma, Hannover, Germany) were added per well of a Polysorb ELISA plate (Nunc, Roskilde, Denmark) and dried overnight at 37° C. Coated wells were washed 3 times with 200 μl PBS+0.02% Tween 20 (Identical washing steps were performed after each incubation step.) followed by 30 minutes incubation with 200 μl of PBS+2% BSA for blocking. After one wash, 50 μl of His-Tagged Subunit B of the toxin Stx1 or Stx2 at indicated concentration were added for 2 hour at room temperature or 37° C. The plate was then washed three times with PBS+0.02% Tween 20 and incubated with 50 μl Anti-His antibody-HRP labelled 1/3000 (Rabbit Mab@His from Biomol or Mouse Mab@His from Biolegend) for 1 hour at room temperature or 37° C. Alternatively, 50 μl of HRP-labelled whole Stx1 or Stx2 were directly added at indicated concentration.

After three final washes, 100 μl of TMB One Component HRP Microwell Substrate (3,3′,5,5′-tetramethylbenzidine, pH 4.5, Tebu-bio, France) was added as substrate, reaction was stopped by addition of 50 μl 2.5 N H₂SO₄ and extinction was measured at E450/630 nm (ELISA Reader, Dynex Technologies Inc., Chantilly, USA).

Optionally, to confirm the carbohydrate specificity of the binding, coated Gb3 may be submitted to enzymatic or chemical treatment affecting the structure of the carbohydrate. As an example, mild (carbohydrate-specific) periodate oxidation (PI) was used as described in example 1 prior to incubation with the Shiga toxin. The plates were washed five times and the ELISA was carried out as described above.

Procedure for Testing the Capacity of Labeled Toxin to Bind its Natural Receptor on the Surface of Human Cells.

Cytotoxic Cellular Assay

Human colorectal adenocarcinoma cell line HT-29 were seeded in 96 well cell culture plates at 5×10³ to 1×10⁴ cells/well and cultivated for 2 days in standard McCoy Medium added with 10% FBS. 20 μl of a solution of Shiga Toxin 1 or 2 at indicated concentration labeled or not were added per well and incubated for 30 hours at 37° C. The cellular survival rate was then analyzed by mean of an MTT-Assay.

Isolation of Microorganism Naturally Binding Shiga Toxin 1 or Shiga Toxin 2

Isolation of bacteria using toxin-coated beads was performed as described in example 2.

Growth, inactivation, maintenance and characterization of isolates were performed as described in example 2.

Screening of Bacterial Strains Binding the Shiga Toxin 1 or 2

Procedure for Testing the Capacity of Isolates to Bind the Shiga Toxin 1 or 2

Standardization of assays and coating of the Polysorp microtiterplate (Nunc, Roskilde, Denmark) were performed as described in Example 1.

Plates were washed 3 times with PBS+0.02% Tween 20. Plates were blocked with 200 μl PBS+2% Bovine serum albumin (BSA) or with 200 μl PBS+2% Bovine serum albumin (BSA)+appropriate antibody isotype control (Rabbit IgG or Mouse IgG) to block antibody binding proteins potentially present on the surface of the isolates. ELISA plates were then incubated with 50 μl of 200 ng/ml HRP-labelled Shiga toxin (1 or 2) or with His-tagged Shiga toxin (1 or 2) subunit B for 2 hour at room temperature or 37° C. The plates were then washed three times with PBS+0.02% Tween 20 and further incubated for 1 hour at room temperature with an anti-His antibody (as described above) or directly developed with TMB as substrate. The reaction was stopped by addition of 2.5 N H₂SO₄ and extinction was measured at 450/630 nm (as detailed above).

To confirm the involvement of carbohydrate structure(s) in the binding of the Cholera Toxin to isolated microorganisms, coated bacteria were submitted to mild periodate oxidation (PI) as described in Example 1 prior to incubation with heat labile toxin. Stx non-binding strains from diff. species were used as negative control (i.e. Lactobacillus, Bifidobacterium, Enterococcus, Citrobacter etc. . . . ).

Results

Binding Activity of HRP Labelled Shiga Toxins

The ability of HRP labeled Shiga toxin 1 and 2 to bind their natural receptor Gb3 was analyzed by means of an ELISA. As presented in FIG. 8, HRP labeled Shiga toxin 1 bound to Gb3 in a specific and concentration dependent manner. The same results were obtained with the His-Tagged Stx subunit B of the toxins and both anti-His secondary antibodies mentioned above. Furthermore, the mild periodate oxidation of Gb3 prior to incubation with the toxins resulted in a strong reduction of the signal, confirming the carbohydrate specificity of the interaction. Similar results were obtained with HRP labeled Shiga toxin 2 and His-Tagged Stx2 subunit B with the difference that the signals were generally reduced by 30 to 50% in accordance with a higher binding efficiency of Stx1 to Gb3 (49).

Cytotoxicity Activity of HRP-Labeled Shiga Toxin

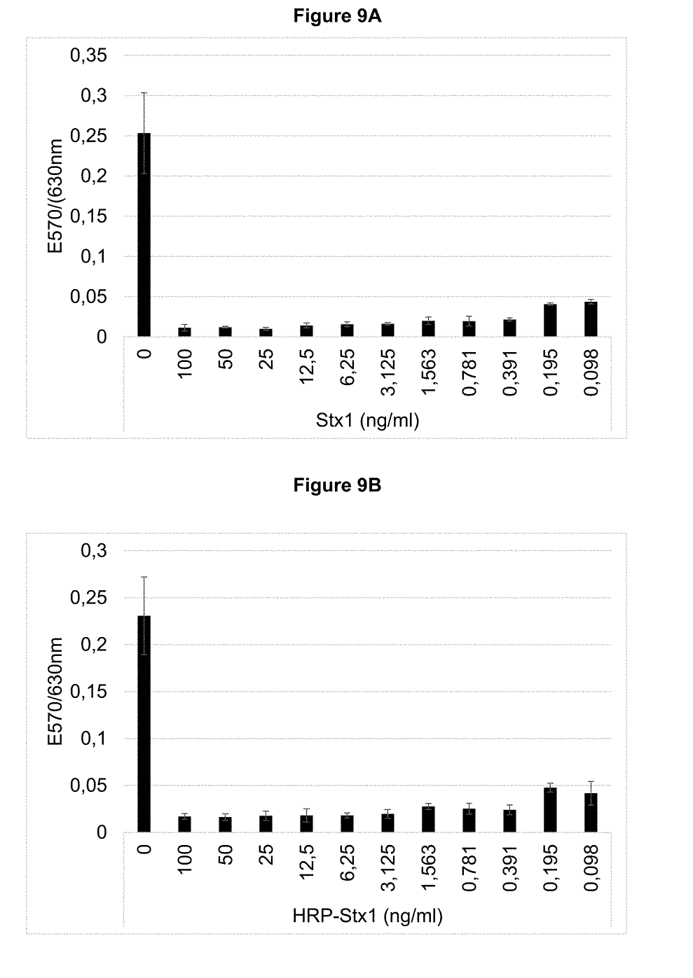

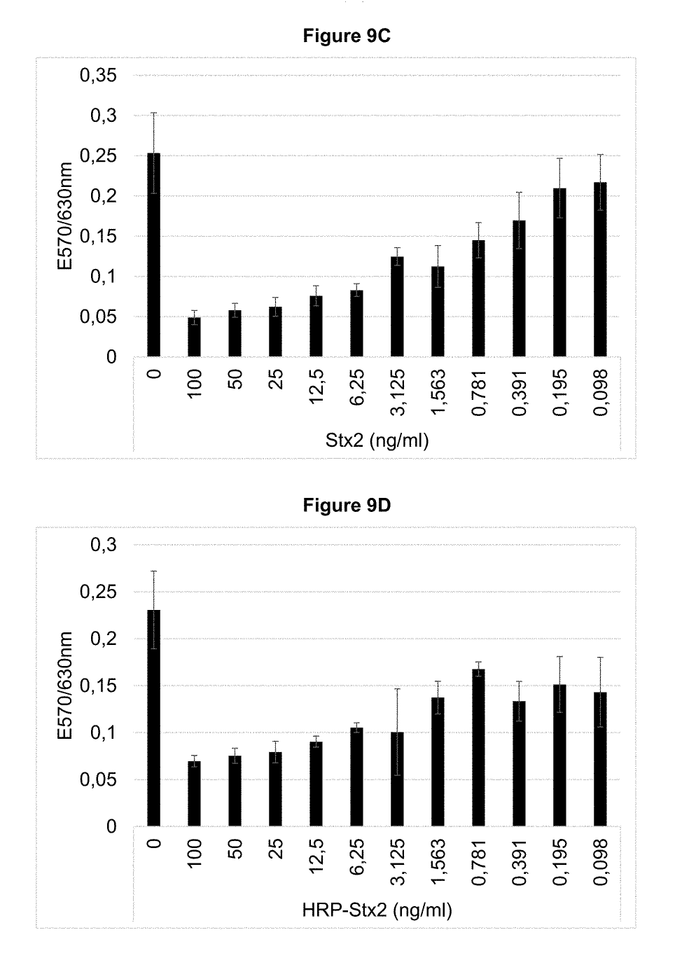

The ability of unlabeled and HRP-labelled Stx1 and Stx2 Toxins to bind their natural receptor on the surface of HT-29 cells and to induce cytotoxicity was analyzed by mean of MTT-Assay. Unlabeled and HRP-labeled Stx1 and Stx2 toxins presented identical concentration dependent cytotoxicity effect on HT-29 cells, demonstrating that HRP-labeled Shiga toxins conserved their ability to bind their natural receptor on the surface of HT29 cells and to induce cytotoxicity (FIG. 9).

Binding of Shiga Toxins and Periodate Sensitivity

The capacity of isolated commensal bacteria to bind Shiga toxin 1 or 2 by means of an enzyme-linked immunosorbent assay (ELISA) using HRP-labeled Shiga toxins and His-tagged Shiga toxins subunit B was analyzed.

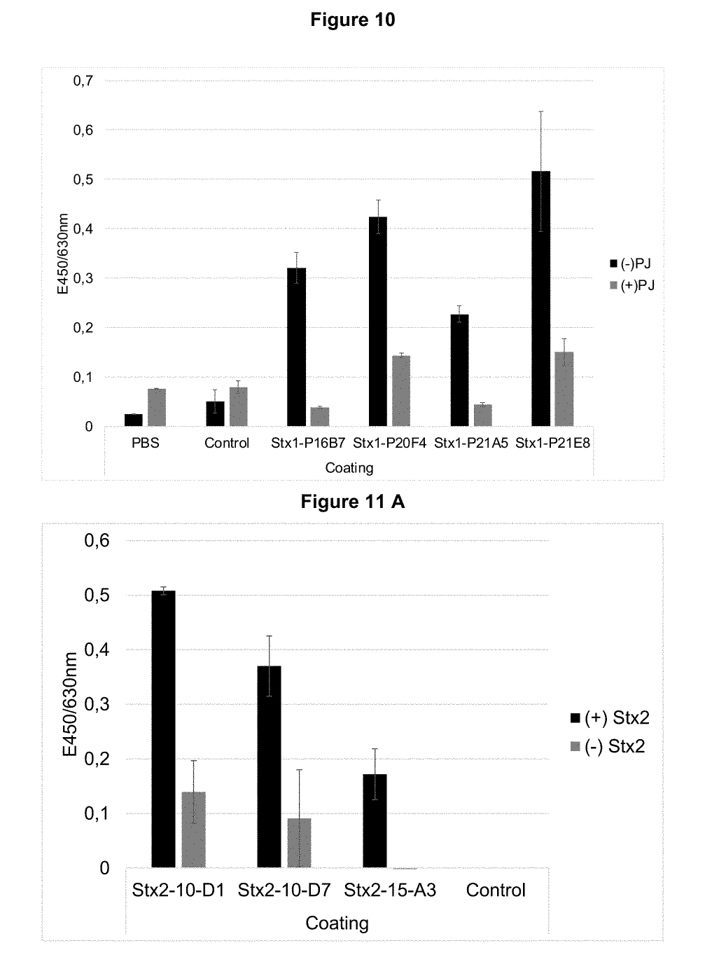

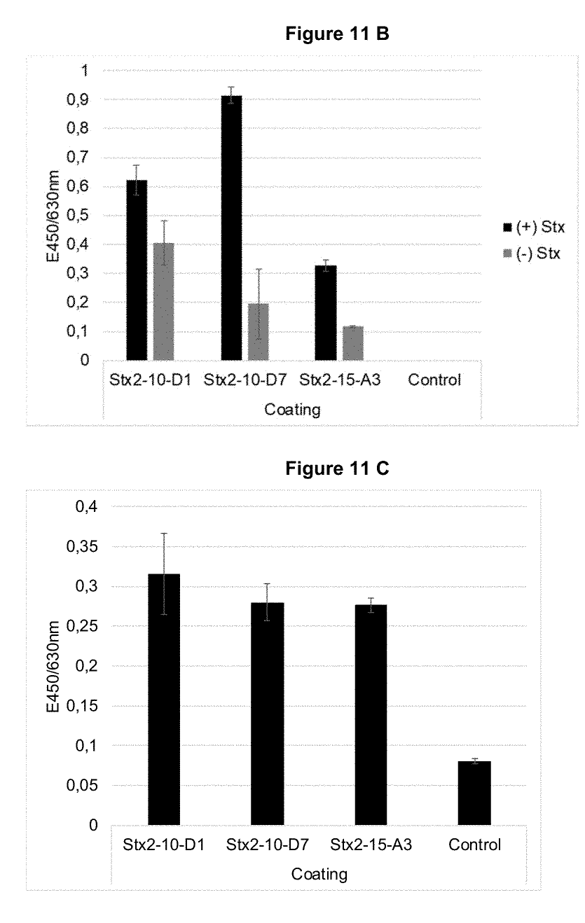

Four strains presented a strong and dose dependent natural binding capacity for Shiga Toxin 1. Furthermore, for all four isolates the binding was reduced by prior mild periodate oxidation of the coated bacteria, confirming the involvement of a carbohydrate structure in the binding of Shiga Toxin 1 (FIG. 10). Furthermore, three strains presented a natural binding capacity for Shiga Toxin 2 (FIG. 11).

Interestingly, the strains Stx1-P21E8 and Stx2-15-A3 were able to bind both toxins Stx1 and Stx2.

The Stx1 binding strain STx1-P16B7 was characterized as belonging to the species citrobacter freundii, the strains Stx1-P20F4, Stx1-P21A5 and Stx1-P21E8 were characterized as belonging to the species klebsiella oxytoca (see table 1). The Stx2 binding strains STx2-10D1, Stx2-10-D7 and Stx2-15-A3 were characterized as belonging to the species citrobacter freundii and Enterococcus feacalis (see table 1).

Conclusion

The present study is the first one to report the isolation out of the human gut microflora of microorganisms naturally expressing a specific binding moiety for the Shiga toxin 1 and Shiga toxin 2. The binding of the toxins was sensitive to mild periodate oxidation, suggesting that the binding moiety expressed on the surface of the bacteria contains a carbohydrate structure directly involved in the binding.

These surprising results unequivocally demonstrate that the invention allows isolating natural inhabitants of the human flora that are naturally expressing a binding moiety for the shiga toxin 1 and 2. Such microorganisms may be develop as drug or food supplement to be administered orally to human or animal in viable or killed form to abate, cure, treat or prevent diseases related with Shiga toxin.

### REFERENCES

1. Ofek I, Hasty D. L, Doyle R. J. (2003): Bacterial Adhesion to Animal Cells and Tissues. American Society for Microbiology, DC, USA
2. Cozens D, Read R. C. (2012): Anti-adhesion methods as novel therapeutics for bacterial infections. Expert Rev Anti Infect Ther. 10(12):1457-1468.
3. Sa E. C, Griffiths N. J., Murillo I., Virji M. (2009): Neisseria meningitidis Cell. Microbiol. 11(3):389-405
4. Holmes A. R., McNab R., Millsap K. W. et al. (2001): The pavA gene of Streptococcus pneumonia encodes a fibronectin-binding protein that is essential for virulence. Mol. Microbiol. 41(6):1395-1408
5. Cundell D. R., Gerard N. P., Gerard C., Idanpaan-Heikkila I., Tuomanen E. I. (1995): Streptococcus pneumoniae anchor to activated human cells by the receptor for platelet-activating factor. Nature 377(6548):435-438
6. Imberty A. and Varrot A. (2008): Microbial recognition of human cell surface glycoconjugates. Curr. Opin. Struct. Biol. 18(5): 567-576.
7. Pieters R. J. (2011): Carbohydrate mediated bacterial adhesion. Adv. Exp. Med. Biol. 715: 227-240
8. Bernardi A., Jiménez-Barbero J., Casnati A., De Castro C., Darbre T., Fieschi F., et al. (2012): Multivalent glycoconjugates as anti-pathogenic agents. Chem Soc Rev. 42(11):4709-27.
9. U.S. Pat. No. 6,833,130
10. Nagahama M., Oda M., Kobayashi K., Ochi S., Takagishi T., Shibutani M., Sakurai J. (2013): A recombinant carboxy-terminal domain of alpha-toxin protects mice against Clostridium perfringens. Microbiol Immunol. 57(5):340-345
11. Focareta A., Paton J. C., Morona R., Cook J., Paton A. W. (2006): A recombinant probiotic for treatment and prevention of cholera. Gastroenterology. 130(6):1688-1695. Focareta A et al,
12. Mally M., Fontana C., Leibundgut-Landmann S., Laacisse L., Fan Y. Y., Widmalm G., Aebi M. (2013): Glycoengineering of host mimicking type-2 LacNAc polymers and Lewis X antigens on bacterial cell surfaces. Mol Microbiol. 87(1):112-131.
13. U.S. Pat. No. 8,192,978
14. US20070148149
15. WO2012152270A1
16. Houliston R. S., Vinogradov E., Dzieciatkowska M., Li J., St Michael F., Karwaski M. F., Brochu D., et al. (2011): Lipooligosaccharide of Campylobacter jejuni—similarity with multiple types of mammalian glycans beyondgangliosides. J Biol Chem. 286(14):12361-12370
17. Yuki N. (2010): Human Gangliosides and Bacterial Lipo-oligosaccharides in the Development of Autoimmune Neuropathies. Methods Mol Biol. 600:51-65.
18. Martin S. L., Edbrooke M. R., Hodgman T. C., van den Eijnden D. H., Bird M. I. (1997): Lewis X Biosynthesis in Helicobacter pylori. Molecular cloning of an alpha(1,3)-fucosyltransferase gene. J Biol Chem. 272(34):21349-21356.
19. Van Remoortere A., Hokke C. H., van Dam G. J., van Die I., Deelder A. M., van den Eijnden D. H. (2000): Various stages of schistosomas express Lewis(x), LacdiNAc, GalNAcG1-4(Fucalpha1-3)GlcNAc and GalNAcbeta1-4(Fucalpha1-2Fucalpha1-3)GlcNAc carbohydrate epitope: detection with monoclonal antibodies that are characterized by enzymatically synthesized neopglycoproteins. Glycobiology. 10(6):601-609.
20. Leffler D. A., Lamont J. T. (2012): Editorial: not so nosocomial anymore: the growing threat of community-acquired Clostridium difficile. Am J Gastroenterol 107:96-98
21. Miller B. A., Chen L. F., Sexton D. J., Anderson D. J. (2011): Comparison of the Burdens of Hospital-Onset, Healthcare Facility-Associated Clostridium difficile Infection and of Healthcare-Associated Infection due to Methicillin-Resistant Staphylococcus aureus in Community Hospitals. Infect Control Hosp Epidemiol. 32(4):387-290.
22. Hookman P. and Barkin J. S. (2009): Clostridium difficile associated infection, diarrhea and colitis. World J Gastroenterol. 15(13):1554-1580
23. Vaishnavi C. (2010): Clinical spectrum & pathogenesis of Clostridium difficile associated diseases. Indian J Med Res. 131:487-499
24. Sun X., Savidge T. and Feng H. (2010): The Enterotoxicity of Clostridium difficile Toxins. Toxins (Basel). 2(7):1848-1880.
25. Pruitt R. N., Chambers M. G., Ng K. K., Ohi M. D. and Lacy D. B. (2010): Structural organization of the functional domains of Clostridium difficile toxins A and B. Proc Natl Acad Sci USA. 107(30):13467-13472.
26. Jank T., Giesemann T., and Aktories K. (2007): Rho-glucosylating Clostridium difficile toxins A and B: new insights into structure and function. Glycobiology 17(4):15-22.
27. Voth D. E. and Ballard J. D. (2005): Clostridium difficile Toxins: Mechanism of Action and Role in Disease. Clin Microbiol Rev. 18(2):247-263.
28. Oiling A., Goy S., Hoffmann F., Tatge H., Just I., Gerhard R. (2011): The Repetitive Oligopeptide Sequences Modulate Cytopathic Potency but Are Not Crucial for Cellular Uptake of Clostridium difficile Toxin A. PLoS ONE 6(3):e17623.
29. Pruitt R. N. and Lacy D. B. (2012): Toward a structural understanding of Clostridium difficile toxins A and B. Front. Cell. Inf. Microbio. 2(28):1-14.
30. Bertholet-Thomas A., Ranchin B., King L. A., Bacchetta J., Belot A., Gillet Y., Collardeau-Frachon S., Cochat P. (2011): Post-diarrheal haemolytic uremic syndrome: when shall we consider it? Which follow-up?. Arch Pediatr. 18(7):823-830.
31. Oakes R. S., Kirkhamm J. K., Nelson R. D., Siegler R. L. (2008): Duration of oliguria and anuria as predictors of chronic renal-related sequelae in post-diarrheal hemolytic uremic syndrome. Pediatric Nephrology, 23(8):1303-1308.
32. Garg A. X., Suri R. S., Barrowman N., Rehman F., Matsell D., Rosas-Arellano M. P., Salvadori M., et al. (2003): Long-term renal prognosis of diarrhea-associated hemolytic uremic syndrome: a systematic review, meta-analysis, and meta-regression. J. Am Medl Assoc. 290(10):1360-1370.
33. Obrig T. G. (2010): Escherichia coli Shiga Toxin Mechanisms of Action in Renal Disease. Toxins (Basel). 2(12):2769-2794.
34. Kosek M., Bern C., Guerrant R. L. (2003): The global burden of diarrhoeal disease, as estimated from studies published between 1992 and 2000. Bull World Health Organ. 81(3):197-204.
35. Steffen R., de Bernardis C., Banos A. (2003): Travel epidemiology—a global perspective. Int J Antimicrob Agents 21(2):89-95.
36. Qadri F., Svennerholm A. M., Faruque A. S., Sack R. B. (2005): Enterotoxigenic Escherichia coli in developing countries: epidemiology, microbiology, clinical features, treatment, and prevention. Clin Microbiol Rev 18(3):465-483.
37. Isidean S. D., Riddle M. S., Savarino S. J., Porter C. K. (2011): A systematic review of ETEC epidemiology focusing on colonization factor and toxin expression. Vaccine. 29(37):6167-6178. 1.
38. Gaastra W., Svennerholm A. M. (1996): Colonization factors of human enterotoxigenic Escherichia coli (ETEC). Trends Microbiol 4(11):444-452.
39. Wolf M. (1997): Occurrence, distribution, and associations of O and H serogroups, colonization factor antigens, and toxins of enterotoxigenic Escherichia coli. Clin Microbiol Rev 10(4):569-584.
40. Jiang Z. D., Lowe B., Verenkar M. P., Ashley D., Steffen R., Tornieporth N., von Sonnenburg F., et al. (2002): Prevalence of enteric pathogens among international travelers with diarrhea acquired in Kenya (Mombasa), India (Goa), or Jamaica (Montego Bay). J Infect Dis 185(4):497-502.
41. Sack D A, Shimko J, Torres O, Bourgeois A L, Francia D S, Gustafsson B, Karnell A, et al. (2007): Randomised, double-blind, safety and efficacy of a killed oral vaccine for enterotoxigenic E. coli diarrhoea of travelers to Guatemala and Mexico. Vaccine 25(22):4392-4400.
42. Minke W. E., Hong F, Verlinde C. L., Hol W. G., Fan E. (1999): Using a galactose library for exploration of a novel hydrophobic pocket in the receptor binding site of the Escherichia coli heat-labile enterotoxin. J Biol Chem. 274(47):33469-33473.
43. Weiglmeier P. R., Rosch P., Berkner H. (2010): Cure and Curse: E. coli Heat-Stable Enterotoxin and Its Receptor Guanylyl Cyclase C. Toxins (Basel) 2(9):2213-2229.
44. Jansson L., Tobias J., Jarefjall C., Lebens M., Svennerholm A. M., et al. (2009): Sulfatide Recognition by Colonization Factor Antigen CS6 from Enterotoxigenic Escherichia coli. PLoS ONE 4(2): e4487.
45. World Health Organization. Cholera. Fact sheet no. 107. Geneva: World Health Organization; 2010 [Available from: http://www.who.int/mediacentre/factsheets/fs107/en/index.html.
46. Clements, J. D., R. J. Yancey, and R. A. Finkelstein. 1980. Properties of homogeneous heat-labile enterotoxin from Escherichia coli. Infect. Immun. 24:91-97.
47. Holmgren, J. 1994. Receptors for cholera toxin and Escherichia coli heat labile enterotoxin revisited. Prog. Brain Res. 101:163-177.
48. Barnard, J. A., and Warwick G. (1993): Butyrate rapidly Induces Growth inhibition and Differentiation in HT-29 cells. Cell Growth & Differentiation Vol. 4, 495-201.
49. Saito M, Mylvaganum M, Tam P, Novak A, Binnington B, et al. (2012) Structure-dependent pseudoreceptor intracellular traffic of adamantyl globotriaosyl ceramide mimics. The Journal of biological chemistry 287: 16073-16087.

1-30. (canceled)

31. A method for isolating a naturally-occurring microorganism that displays a structure (A) on a surface of said microorganism, said structure (A) being capable of binding a toxin from a pathogenic microorganism, comprising:
(a) bringing a composition comprising one or more microorganisms into contact with (i) said toxin and/or (ii) a first binding moiety being capable of binding said structure (A); and
(b) obtaining one or more microorganisms bound by said toxin and/or said first binding moiety.

32. A method for isolating a naturally-occurring microorganism that displays a structure (B) on a surface of said microorganism, said structure (B) being capable of binding a surface receptor of a mammalian cell for a toxin from a pathogenic microorganism, comprising:
(a) bringing a composition comprising one or more microorganisms into contact with (iii) said surface receptor and/or (iv) a second binding moiety being capable of binding said structure (B); and
(b) obtaining one or more microorganisms bound by said surface receptor and/or said second binding moiety.

33. The method of claim 31, wherein said first binding moiety is an antibody, lipocalin or lectin.

34. The method of claim 31, comprising culturing said obtained microorganism.

35. The method of claim 31, comprising testing said obtained microorganism for a capacity to (i) neutralize the toxin and/or (ii) reduce the pathogenicity of a pathogenic microorganism.

36. The method of claim 31, wherein said microorganism that displays said structure (A) on the surface is a bacterium, yeast or fungus.

37. The method of claim 31, wherein said microorganism comprised in said composition is a bacterium, yeast, fungus, virus, or protozoan organism.

38. The method of claim 31, wherein said structure (A) comprises a protein, glycoprotein, lipid, glycolipid or carbohydrate structure.

39. The method of claim 31, wherein said toxin is Heat labile toxin (LT), Heat stabile toxin (ST), Verotoxins/Shiga like toxins (Stxs), Cytotoxins, endotoxins (LPS), EnteroAggregative ST toxin (EAST), Shiga toxin (STxs), Shigella enterotoxins 1 (ShET1), Shigella enterotoxins 2 (ShET2), Neurotoxin, Cytolethal distending toxins (Cdt), AvrA toxin, Cytotoxic necrotizing facto (CNFy), Yersinia murine toxin (Ymt), Yst toxin, Toxin complex (TCa), Heat stabile toxin, E. cloacae leukotoxin, Shiga-like toxin II, heat-stable like enterotoxins, extracellular toxic complex (ETC), Hemolysins (Shl), Pore-forming Toxin (PFT), α-hemolysin (HlyA), heat-stable like toxin, Cytotoxins, C. perfringens alpha-toxin (CpPLC), C. perfringens beta toxin, C. perfringens enterotoxin (CPE), C. difficile enterotoxins (Tcd), C. butulinum Neurotoxins, C. tetani Tetanospasmin, C. butulinum C2 toxin, C. butulinum C3 toxin, C. perfringens epsilon-toxin (e-toxin), C. perfringens iota-toxin (i-toxin), tetanus neurotoxin (TeNT), theta-toxin/PFO (perfringolysin O), C. spiroforme (spiroforme toxin), C. septicum (a-toxin), Lecithinase, Cholera toxins (CTx), accessory cholera enterotoxin (Ace), RTX toxin, zona occludens toxin (Zot), Cholix toxin, α-hemolysin, β-hemolysin, δ-hemolysin, γ-hemolysin, Exfoliative toxins (Exofoliatins), Panton-Valentine leukocidin (PVL), staphylococcal enterotoxins (SE), Toxic shock syndrome toxin-1 (TSST-1), β-haemolysin/cytolysin, CAMP factor, Streptolysin O, Streptolysin S, Pneumolysin, S. pyogenes Exotoxins (PSE), vacuolating cytotoxin A (VacA), Cytolytic toxins, Exotoxins (ex: ExoA, ExoS, ExoT, ExoU, ExoY), Phospholipase C (PLC), Pasteurella Multocida Toxin (PMT), RTX toxins, B. weihenstephanensis endotoxins, B. cereus Hemolysin BL (Hbl), B. cereus, onhemolytic Enterotoxin (Nhe), B. cereus Cytotoxin K (CytK), B. cereus emetic toxin, B. cereus toxin (Cereolysin), B. anthracis (Anthrax toxin), B. thuringiensis δ-,endotoxins (Cry toxins), Cytolethal distending toxin (cdtA, cdtB, cdtC), cholera-like enterotoxin, Aerolysin Cytotoxic Enterotoxin (ACT), ADP-ribosylation toxin, a-hemolysins, b-hemolysins, Heat labile toxin (LT+), Heat stabile toxin (ST+), endotoxins (LPS), B. pertussis (pertusis toxin), Adenylate cyclase toxin, Tracheal cytotoxin, Dermonecrotic (heat-labile) toxin, endotoxins (LPS), Endotoxin (LOS), Cytolethal distending toxins (HdCDT), Hemolysins, Endotoxins, Cytotoxins, Diphteria toxin, Exotoxins, Bacteroides fragilis toxin (bft), Listeriolysin O, or rota virus toxin (NSP4).

40. The method of claim 31, wherein said pathogenic microorganism causes a gastrointestinal disease.

41. The method of claim 31, wherein said toxin and/or said first binding moiety is coupled to a label, a tag, an antibody and/or a bead.

42. The method of claim 41, wherein said antibody is coupled to a bead.

43. The method of claim 31, wherein said composition is from a human sample, animal sample, soil, water, food or culture of microorganisms.

44. The method of claim 31, further comprising admixing said obtained microorganism with a pharmaceutically acceptable carrier.

45. The method of claim 31, wherein said composition comprises one or more microorganisms that are comprised in human or animal feces.

46. A composition comprising a microorganism obtainable by the method of claim 31, wherein said composition is administered by enteral application, and wherein the microorganism is non-pathogenic.

47. A method of treating, alleviating, or preventing a gastrointestinal disease, the method comprising administering an effective amount of the composition of claim 46 to a subject in need thereof.

48. The method of claim 47, wherein said gastrointestinal disease is a gastrointestinal infection.

49. The method of claim 48, wherein said gastrointestinal infection is caused by a bacterium, yeast, fungus, virus, or protozoan organism.

50. The composition of claim 46, wherein the composition is a pharmaceutical composition.

51. A pharmaceutical composition comprising a microorganism obtainable by the method of claim 31 for use in a nutrition of non-human animals.

52. The pharmaceutical composition of claim 51, wherein said composition is administered by enteral application.

53. A microorganism obtainable by the method of claim 31, wherein said microorganism is capable of (i) binding a toxin from a pathogenic microorganism and/or (ii) binding a surface receptor of a mammalian cell for a toxin from a pathogenic microorganism for use in a method of treating, alleviating, or preventing a gastrointestinal disease.
